(19) United States
(12) Patent Application Publication
(10) Pub. No.: US 2014/0246041 A1
(43) Pub. Date: Sep. 4, 2014

(54) HAIR-CONDITIONING AGENTS

(71) Applicant: Henkel AG & Co. KGaA, Duesseldorf (DE)

(72) Inventor: Marcus Krueger, Ellerhoop (DE)

(73) Assignee: Henkel AG & Co. KGaA, Duesseldorf (DE)

(21) Appl. No.: 14/278,421

(22) Filed: May 15, 2014

Related U.S. Application Data
(63) Continuation of application No. PCT/EP2012/070659, filed on Oct. 18, 2012.

(30) Foreign Application Priority Data
Nov. 23, 2011 (DE) 10 2011 086 924.7

(51) Int. Cl. A61K 8/41; A61Q 5/12; A61K 8/49

(52) U.S. Cl. CPC A61K 8/416; A61K 8/4946; A61Q 5/12
USPC 132/202; 424/70.28

(57) ABSTRACT

Hair treatment agents include selected esterquats with selected care substances and to the use of said agents for the treatment of keratinous fibers.

### FIELD OF THE INVENTION

The present invention generally relates to hair-conditioning agents including selected esterquats with selected care-providing substances, and to the use of said agents for the treatment of keratinic fibers.

### BACKGROUND OF THE INVENTION

The significance of care-providing products having the longest-lasting effect possible is increasing, not least due to severe stress on hair as a result, for example, of coloring or permanent waving, and also as a result of cleaning the hair with shampoos and because of environmental stresses.

The known active agents cannot, however, meet all needs to a sufficient extent. A demand therefore continues to exist for active agents or active-agent combinations for cosmetic agents having good care-providing properties and good biodegradability. In dye- and/or electrolyte-including formulations in particular, a demand exists for additional care-providing active agents that can be incorporated without difficulty into known formulations.

Quaternary ammonium compounds of the mono-, di-, and/or trialkylammonium compound types have been known for some time. One disadvantage of these compounds, however, is their deficient biodegradability. Cationic compounds including at least one ester group (the so-called esterquats) were therefore developed. These exhibit, however, in terms of the softness and feel of wet skin and wet hair, as well as the softness and feel of the skin or hair once it has dried after washing, a feel that is judged to be unpleasantly dull, and is also perceived as audibly “squeaky.”

Cationic imidazolines are known to one skilled in the art as a further class of cationic surfactants, for example from the international application WO 2006/012930. The imidazolines as well, however, are not capable of furnishing all the properties expected of an agent providing care to hair.

EP 951 898 B1, furthermore, discloses hair-conditioning agents that include a quaternary ammonium compound from the group of the esterquats, and at least one silicone compound.

Diesterquats, i.e. quaternary ammonium compounds having two acyl residues in the molecule, are disclosed e.g. in EP 918 743.

WO 2004/093834 A1 discloses special diesterquats that are based on diols rather than on ethanolamines, and that comprise long-chain acyl residues that can be bound optionally via PEG or PPG groupings to the diol groupings.

It is therefore desirable to provide hair treatment agents that combine improved care-providing effects, in particular improved combability values, softness, and shine for the hair, with viscosity stability over broad temperature ranges.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

A hair-conditioning agent that includes, based in each case on its total weight, in a cosmetic carrier, a) at least one esterquat according to formula (Tkat1-2)

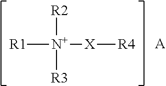

in a total quantity from 0.01 to 20.0 wt %, wherein the residues R1, R2, and R3 are each mutually independent and can be identical or different, and the residues R1, R2, and R3 signify a branched or unbranched alkyl residue having 1 to 4 carbon atoms, in particular methyl, ethyl, propyl, isopropyl, butyl, tert-butyl, or isobutyl, particularly preferably methyl; the residue (—X—R4), provided that at most two of the residues R1, R2, or R3 can denote this residue; the residue —(X—R4) is included at least once and at most twice, particularly preferably twice, and X denotes: —(CH2)n-, where n=1 to 20, preferably n=1 to 10, and particularly preferably n=1 to 5 and most preferably n=2, and R4 denotes R6-O—CO—, in which R6 is a saturated or unsaturated, branched or unbranched, or a cyclic saturated or unsaturated alkyl residue having 6 to 30 carbon atoms, which can include at least one hydroxy group, preferably a saturated, branched or unbranched alkyl residue having 12 to 30 carbon atoms, particularly preferably a saturated unbranched alkyl residue having 12 to 30, preferably 12 to 24 carbon atoms; and A denotes a physiologically acceptable organic or inorganic anion selected from the halide ions fluoride, chloride, bromide, iodide, sulfates of the general formula RSO₃⁻, in which R has the meaning of a saturated or unsaturated alkyl residue having 1 to 4 carbon atoms, or anionic residues of organic acids such as maleate, fumarate, oxalate, tartrate, citrate, lactate, or acetate; and b) at least one care-providing substance(s) in a total quantity of said care-providing substances from 0.001 to 10 wt %, preferably 0.005 to 7.5 wt %, particularly preferably 0.01 to 5 wt %, and in particular 0.05 to 2.5 wt %, wherein the care-providing substance(s) are selected from the group L-carnitine and/or salts thereof, and/or taurine and/or salts thereof, and/or vitamins and vitamin precursors, and/or niacinamide, and/or ubiquinone, and/or ectoin.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now been found that the combination of cationic care-providing substances and specific selected care-providing substances can achieve the aforesaid advantages.

The use of these combinations results in surprisingly good properties for the treated skin and hair, in particular in improved combability values, improved shine, and improved elasticity, as well as an appreciable increase in the washing resistance of colored hair, and in longer durability simultaneously with better reshaping performance in the context of waving operations such as water waving and permanent waving.

A first subject of the present invention is therefore a composition for treating keratinic fibers including, in a cosmetic carrier:
a) at least one esterquat according to formula (Tkat1-2) in a total quantity from 0.01 to 20.0 wt % based on the weight of the total composition, and
b) at least one care-providing substance in a total quantity of said care-providing substances from 0.001 to 10 wt %, preferably 0.005 to 7.5 wt %, particularly preferably 0.01 to 5 wt %, and in particular 0.05 to 2.5 wt %, based on the weight of the total composition, wherein the care-providing substance(s) are selected from the group
   i. L-carnitine and/or salts thereof, and/or
   ii. taurine and/or salts thereof, and/or
   iii. vitamins and vitamin precursors, and/or
   iv. niacinamide, and/or
   v. ubiquinone, and/or
   vi. ectoin.

Ingredients a) and b) are described in detail below. When “active agent complex (A)” is discussed hereinafter, this refers to the ingredients a) and b) obligatorily included in the agents according to the present invention.

“Hair treatment agents” for purposes of the present invention are, for example, hair coloring agents, hair-bleaching agents, hair shampoos, hair conditioners, conditioning shampoos, hair sprays, hair rinses, hair treatments, hair packs, hair tonics, permanent-wave setting solutions, hair coloring shampoos, hair coloring agents, hair setting agents, hair setting compositions, hair styling preparations, blow-dry lotions, foam setting agents, hair gels, hair waxes, or combinations thereof.

“Combability” is understood according to the present invention as both the combability of the wet fibers and the combability of the dry fibers. The combing work expended, or the force expended, during the operation of combing an assemblage of fibers serves as an indication of combability. The measurement parameters can be assessed in sensory fashion by one skilled in the art, or quantified using measurement devices.

“Softness” is defined as the tactility of an assemblage of fibers, in which context one skilled in the art sensorially feels and evaluates the “fullness” and “suppleness” parameters of the assemblage.

“Shapability” is understood as the ability to impart a change in shape to an assemblage of previously treated keratin-including fibers, in particular human hairs. The term “stylability” is also used in hair cosmetics.

An “oxidative hair treatment” is defined according to the present invention as the action on hair of an oxidative cosmetic agent including, in a cosmetic carrier, at least one oxidizing agent.

Suitable according to the present invention as cosmetic carriers are, in particular, O/W, W/O, and W/O/W emulsions in the form of creams or gels or also surfactant-including foaming solutions such as, for example, shampoos, foam aerosols or other preparations, that are suitable in particular for application to the hair. It is also conceivable, however, to integrate the ingredients into a powdered or also tablet-shaped formulation that is dissolved in water prior to utilization. The cosmetic carriers can be, in particular, aqueous or aqueous alcoholic.

An “aqueous” cosmetic carrier includes at least 50 wt % water.

“Aqueous alcoholic” cosmetic carriers are to be understood for purposes of the present invention as aqueous solutions including 3 to 70 wt % of a C₁ to C₆ alcohol, in particular methanol, ethanol, or propanol, isopropanol, butanol, isobutanol, tert-butanol, n-pentanol, isopentanols, n-hexanol, isohexanols, glycol, glycerol, 1,2-pentanediol, 1,5-pentanediol, 1,2-hexanediol, or 1,6-hexanediol. The agents according to the present invention can additionally include further organic solvents such as, for example, methoxybutanol, benzyl alcohol, ethyl diglycol, or 1,2-propylene glycol. All water-soluble organic solvents are preferred in this context.

The agents according to the present invention include as ingredient a) at least one esterquat having the structure (Tkat1-2) depicted below:

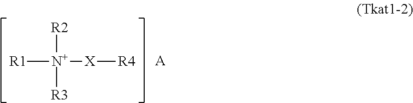

in which the residues R1, R2, and R3 are each mutually independent and can be identical or different. The residues R1, R2, and R3 signify:
a branched or unbranched alkyl residue having 1 to 4 carbon atoms, in particular methyl, ethyl, propyl, isopropyl, butyl, tert-butyl, or isobutyl, particularly preferably methyl,
the residue (—X—R4), provided that at most two of the residues R1, R2, or R3 can denote this residue.

The residue —(X—R4) is included at least once and at most twice, particularly preferably twice.

X herein denotes:
1) —(CH2)ₙ—, where n=1 to 20, preferably n=1 to 10, and particularly preferably n=1 to 5 and most preferably n=2,
   and R4 denotes
1) R6-O—CO—, in which R6 is a saturated or unsaturated, branched or unbranched, or a cyclic saturated or unsaturated alkyl residue having 6 to 30 carbon atoms, which can include at least one hydroxy group, preferably a saturated, branched or unbranched alkyl residue having 12 to 30 carbon atoms, particularly preferably a saturated unbranched alkyl residue having 12 to 30, preferably 12 to 24 carbon atoms,
   and A denotes a physiologically acceptable organic or inorganic anion. The anion of all cationic compounds described is selected from the halide ions fluoride, chloride, bromide, iodide, sulfates of the general formula RSO₃⁻, in which R has the meaning of a saturated or unsaturated alkyl residue having 1 to 4 carbon atoms, or anionic residues of organic acids such as maleate, fumarate, oxalate, tartrate, citrate, lactate, or acetate.

Preferred raw materials have a methyl group for R1 and R2 and possess two —X—R4 groups, in which —X—R4-X— respectively denotes ethylene and R4 respectively denotes lauroyl, myristoyl, palmitoyl, stearoyl, arachidoyl, behenoyl, and A denotes chloride or methosulfate. Preferred compounds therefore carry the INCI names Dilauroyl Dimonium Chloride, Dimyristoyl Dimonium Chloride, Dipalmitoyl Dimonium Chloride, Distearoyl Dimonium Chloride, Diarachidoyl Dimonium Chloride, and Dibehenoyl Dimonium Chloride, or the respective methosulfates. A highly preferred raw material having this structure is obtainable commercially under the name Varisoft® EQ 65.

The esterquats of formula (Tkat1-2) are included in the compositions according to the present invention in quantities from 0.01 to 20 wt %, preferably in quantities from 0.01 to 10 wt %, and very particularly preferably in quantities from 0.1 to 7.5 wt %. The best results of all are obtained with quantities from 0.1 to 5 wt %, based in each case on the total composition of the respective agent.

Hair treatment agents according to the present invention are characterized in that they include at least one care-providing substance b) in a total quantity from 0.001 to 10 wt %, preferably 0.005 to 7.5 wt %, particularly preferably 0.01 to 5 wt %, and in particular 0.05 to 2.5 wt %, based in each case on the total weight of the composition, wherein the care-providing substance(s) are selected from the group
i. L-carnitine and/or salts thereof, and/or
ii. taurine and/or salts thereof, and/or
iii. vitamins and vitamin precursors, and/or
iv. niacinamide, and/or
v. ubiquinone, and/or
vi. ectoin.

L-carnitine (IUPAC name: (R)-(3-carboxy-2-hydroxypropyl)-N,N,N-trimethylammonium hydroxide) is a naturally occurring vitamin-like substance.

As a betaine, L-carnitine can form addition compounds and double salts. L-Carnitine derivatives preferred according to the present invention are selected in particular from acetyl-L-carnitine, L-carnitine fumarate, L-carnitine citrate, lauroyl-L-carnitine, and particularly preferably L-carnitine tartrate. The aforesaid L-carnitine compounds are obtainable, for example, from Lonza GmbH (Wuppertal, Germany).

Preferred hair treatment agents according to the present invention are characterized in that they include, based on their weight, 0.001 to 10 wt %, preferably 0.005 to 7.5 wt %, particularly preferably 0.01 to 5 wt %, and in particular 0.05 to 2.5 wt % L-carnitine or L-carnitine derivatives, preferred L-carnitine derivatives being selected from acetyl-L-carnitine, L-carnitine fumarate, L-carnitine citrate, lauroyl-L-carnitine, and in particular L-carnitine tartrate.

A further care-providing substance that is preferred for use, which possesses activating properties, is taurine. Hair treatment agents preferred according to the present invention include, based on their weight, 0.01 to 15 wt %, preferably 0.025 to 12.5 wt %, particularly preferably 0.05 to 10 wt %, more preferably 0.1 to 7.5 wt %, and in particular 0.5 to 5 wt % taurine (2-aminoethanesulfonic acid).

A further preferred group of care-providing substances in the agents according to the present invention is vitamins, provitamins, or vitamin precursors. These are described below:

The group of substances referred to as “vitamin A” includes retinol (vitamin A₁) as well as 3,4-didehydroretinol (vitamin A₂). β-Carotene is the provitamin of retinol. Vitamin A components that are suitable according to the present invention are, for example, vitamin A acid and esters thereof, vitamin A aldehyde, and vitamin A alcohol, as well as esters thereof such as the palmitate and the acetate. The agents according to the present invention include the vitamin A component preferably in quantities from 0.05 to 1 wt %, based on the total preparation.

Members of the vitamin B group or vitamin B complex are, among others:
Vitamin B₁ (thiamine)
Vitamin B₂ (riboflavin)
Vitamin B₃. The compounds nicotinic acid and nicotinic acid amide (niacinamide) are often listed under this designation. Nicotinic acid amide is preferred according to the present invention; it is included in the agents used according to the present invention preferably in quantities from 0.05 to 1 wt % based on the total agent.
Vitamin B₅ (pantothenic acid, panthenol, and pantolactone). In the context of this group, panthenol and/or pantolactone are preferably used (see below). Derivatives of panthenol that are usable according to the present invention are, in particular, the esters and ethers of panthenol as well as cationically derivatized panthenols. Individual representatives are, for example, panthenol triacetate, panthenol monoethyl ether and the monoacetate thereof, as well as cationic panthenol derivatives disclosed in WO 92/13829. The aforesaid compounds of the vitamin B₅ type are included in the agents according to the present invention preferably in quantities from 0.05 to 10 wt % based on the total agent. Quantities from 0.1 to 5 wt % are particularly preferred.
Vitamin B₆ (pyridoxine as well as pyridoxamine and pyridoxal).

Vitamin C (ascorbic acid). Vitamin C is utilized in the agents according to the present invention preferably in quantities from 0.1 to 3 wt % based on the total agent. Utilization in the form of the palmitic acid ester, glucosides, or phosphates can be preferred. Utilization in combination with tocopherols can likewise be preferred.

Vitamin E (tocopherols, in particular α-tocopherol). Tocopherol and its derivatives, which include in particular esters such as the acetate, nicotinate, phosphate, and succinate, are included in the agents according to the present invention preferably in quantities from 0.05 to 1 wt % based on the total agent.

Vitamin F. The term “vitamin F” is usually understood to mean essential fatty acids, in particular linoleic acid, linolenic acid, and arachidonic acid.

Vitamin H. “Vitamin H” refers to the compound (3aS,4S,6aR)-2-oxohexahydrothienol[3,4-d]-imidazole-4-valeric acid, for which the trivial name “biotin” has, however, now become established. Biotin is included in the agents according to the present invention preferably in quantities from 0.0001 to 1.0 wt %, in particular in quantities from 0.001 to 0.01 wt %.

In summary, hair treatment agents according to the present invention that include, based on their weight, 0.1 to 5 wt %, preferably 0.2 to 4 wt %, particularly preferably 0.25 to 3.5 wt %, more preferably 0.5 to 3 wt %, and in particular 0.5 to 2.5 wt % vitamins and/or provitamins and/or vitamin precursors that preferably are assigned to the groups A, B, C, E, F, and H, where preferred agents include -2,4-dihydroxy-N-(3-hydroxypropyl)-3,3-dimethylbutyramide (provitamin B₅) and/or pantothenic acid (vitamin B₃, vitamin B₅) and/or niacin, niacinamide or nicotinamide (vitamin B3) and/or L-ascorbic acid (vitamin C) and/or thiamin (vitamin B₁) and/or riboflavin (vitamin B₂, vitamin G) and/or biotin (vitamin B7, vitamin H) and/or folic acid (vitamin B9, vitamin B_{c} or vitamin M) and/or vitamin B₆ and/or vitamin B₁₂, are preferred.

It has been found that specific quinones have a particular suitability as a care-providing substance. The agents according to the present invention can therefore include, as a further care-providing substance, 0.0001 to 5 wt % of at least one bioquinone of formula (Ubi)

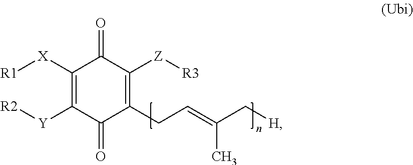

in which
X, Y, Z mutually independently denote —O— or —NH— or —NR⁴⁻ or a chemical bond,
R¹, R², R³ mutually independently denote a hydrogen atom or an optionally substituted aryl group or an optionally substituted (C₁ to C₆) alkyl group or a hydroxyalkyl group or a polyhydroxyalkyl group or an optionally substituted (C₁ to C₆) alkylene group, or a (C₁ to C₆) acyl group, preferred residues being selected mutually independently from —H, —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃,
R⁴ denotes —CH₃, —CH₂CH₃, —(CH₂)₂CH₂, —CH(CH₃)₂, —(CH₂)₃CH₃, —CH(CH₃)CH₂CH₃, —CH₂CH(CH₃)₂, —C(CH₃)₃,
n denotes values from 1 to 20, preferably from 2 to 15, and in particular 5, 6, 7, 8, 9, 10.

Particularly preferred hair treatment agents according to the present invention are characterized in that they include as a care-providing substance, based on their weight, 0.0001 to 1 wt %, preferably 0.001 to 0.5 wt %, and particularly preferably 0.005 to 0.1 wt % of at least one ubiquinone and/or at least one ubiquinol and/or at least one derivative of said substances, wherein preferred agents include a ubiquinone of formula (Ubi)

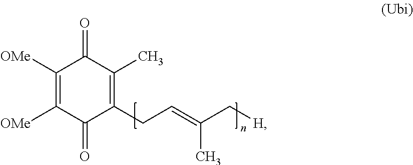

in which n denotes the values 6, 7, 8, 9, or 10, particularly preferably 10 (Coenzyme Q10).

Alternatively or in addition to the particularly preferred ubiquinones, the agents according to the present invention can also include plastoquinones. Preferred agents according to the present invention are here characterized in that they include 0.0002 to 4 wt %, preferably 0.0005 to 3 wt %, particularly preferably 0.001 to 2 wt %, more preferably 0.0015 to 1, and in particular 0.002 to 0.5 wt % of at least one plastoquinone of formula (Ubi-b)

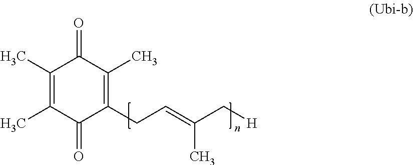

in which n denotes values from 1 to 20, preferably from 2 to 15, and in particular 5, 6, 7, 8, 9, 10, wherein particularly preferred agents include plastoquinone PQ-9 of the formula Ubi-b in which n=9.

As a further care enhancer, the agents according to the present invention can include ectoin. Ectoin ((4S)-2-methyl-1,4,5,6-tetrahydropyrimidine-4-carboxylic acid) is a natural substance belonging to the group of the compatible solutes. This highly water-bonding low-molecular-weight organic compound occurs in halophilic bacteria and allows these extremophile organisms to survive under stress conditions. Hair treatment agents preferred according to the present invention are characterized in that they include, based on their weight, 0.001 to 10 wt %, preferably 0.01 to 5 wt %, particularly preferably 0.05 to 2.5 wt %, and in particular 0.1 to 1 wt % (S)-2-methyl-1,4,5,6-tetrahydro-4-pyrimidinecarboxylic acid (ectoin) as well as the physiologically acceptable salts of that compound, and/or (S,S)-5-hydroxy-2-methyl-1,4,5,6-tetrahydro-4-pyrimidinecarboxylic acid (hydroxyectoin) as well as the physiologically acceptable salts of that compound.

To improve the elasticity and strengthen the internal structure of hair treated with the agents according to the present invention, the agents according to the present invention can include purine and/or purine derivatives as a care-providing substance. In particular, the result of combining purine and/or purine derivatives with ubiquinones and/or plastoquinones as a care-providing substance is that hair treated with corresponding agents exhibits, inter alia, higher measured values in differential thermal analysis, and improved wet and dry combability values.

Purine (7H-imidazo[4,5-d]pyrimidine) does not occur in isolation in nature, but constitutes the basic member of the purines. Purines in turn are a group of important compounds, widespread in nature and involved in human, animal, plant, and microbial metabolic processes, that derive from the basic member by substitution with OH, NH₂, SH in the 2-, 6-, and 8-position, and/or with CH₃ in the 1-, 3-, 7 position. Purine can be manufactured, for example, from aminoacetonitrile and formamide. Purines and purine derivatives are often isolated as natural substances, but are also accessible synthetically by numerous routes.

Preferred agents according to the present invention include purine and/or purine derivatives in narrower quantity ranges. Cosmetic agents preferred according to the present invention are characterized here in that they include, based on their weight, 0.001 to 2.5 wt %, preferably 0.0025 to 1 wt %, particularly preferably 0.005 to 0.5 wt %, and in particular 0.01 to 0.1 wt % purine(s) and/or purine derivative(s).

Among purine, the purines, and the purine derivatives, some representatives are particularly preferred according to the present invention. Hair treatment agents preferred according to the present invention are characterized in that they include as a care-providing substance, based on their weight, 0.001 to 2.5 wt %, preferably 0.0025 to 1 wt %, particularly preferably 0.005 to 0.5 wt %, and in particular 0.01 to 0.1 wt % purine(s) and/or purine derivative(s), wherein preferred agents include purine and/or purine derivative(s) of formula (Pur-I)

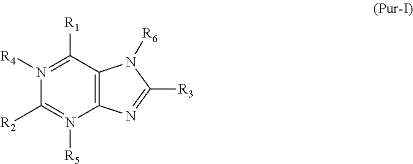

in which the residues R¹, R², and R³ are selected mutually independently from —H, —OH, NH₂, —SH, and the residues R⁴, R⁵, and R⁶ are selected mutually independently from —H, —CH₃, and —CH₂—CH₃, the following compounds being preferred:
purine (R¹=R2=R3=R4=R5=R6=H), adenine (R¹=NH₂, R²=R³=R⁴=R⁵=R⁶=H), guanine (R¹=OH, R²=NH₂, R³=R⁴=R⁵=R⁶=H), uric acid (R¹=R²=R³=OH, R⁴=R⁵=R⁶=H), hypoxanthine (R¹=OH, R²=R3=R⁴=R⁵=R⁶=H), 6-purinethiol (R¹=SH, R²=R³=R⁴=R⁵=R⁶=H), 6-thioguanine (R¹=SH, R²=NH₂, R³=R⁴=R⁵=R⁶=H), xanthine (R¹=R²=OH, R³=R⁴=R⁵=R⁶=H), caffeine (R¹=R²=OH, R³=H, R⁴=R⁵=R⁶=CH₃), theobromine (R¹=R²=OH, R³=R⁴=H, R⁵=R⁶=CH₃), theophylline=R²=OH, R³=H, R⁴=CH₃, R⁵=CH₃, R⁶=H).

It is further advantageous to use purine or purine derivatives and bioquinones at a specific ratio to one another. Preferred in this context are agents according to the present invention in which the weight ratio of purine (derivative(s)) to bioquinone(s) is equal to 10:1 to 1:100, preferably 5:1 to 1:50, particularly preferably 2:1 to 1:20, and in particular 1:1 to 1:10.

As already mentioned, caffeine is a particularly preferred purine derivative and Coenzyme Q10 is a particularly preferred bioquinone. Particularly preferred agents according to the present invention are therefore characterized in that they include, based on their weight, 0.001 to 2.5 wt %, preferably 0.0025 to 1 wt %, particularly preferably 0.005 to 0.5 wt %, and in particular 0.01 to 0.1 wt % caffeine, and 0.0002 to 4 wt %, preferably 0.0005 to 3 wt %, particularly preferably 0.001 to 2 wt %, more preferably 0.0015 to 1, and in particular 0.002 to 0.5 wt % Coenzyme Q10.

The agents according to the present invention can also include flavonoids as a care-providing substance. The flavonoids are a group of water-soluble vegetable dyes and play an important role in the metabolism of many plants. Together with the phenolic acids, they belong to the polyphenols. Well over 6,500 different flavonoids are known, and can be subdivided into flavonols, flavones, flavanones, isoflavanoids, and anthocyans.

Flavonoids from all six groups can be used according to the present invention, specific representatives from the individual groups being preferred as a care-providing substance because of their particularly intense effect. Preferred flavonols are quercetin, rutin, camphor oil, myricetin, isorhamnetin, preferred flavanols are catechin, gallocatechin, epicatechin, epigallocatechin gallate, theaflavin, thearubigin, preferred flavones are luteolin, apigenin, morin, preferred flavanones are hesperetin, naringenin, eriodictyol, preferred isoflavanoids are genistein, daidzein, and preferred anthocyanidins (anthocyans) are cyanidin, delphinidin, malvidin, pelargonidin, peonidin, petunidin.

Hair treatment agents particularly preferred according to the present invention are characterized in that they include, based on their weight, 0.001 to 2.5 wt %, preferably 0.0025 to 1 wt %, particularly preferably 0.005 to 0.5 wt %, and in particular 0.01 to 0.1 wt % flavonoids, in particular flavonols, particularly preferably 3,3′,4′,5,7-pentahydroxyflavone (quercetin) and/or 3,3′,4′,5,7-pentahydroxyflavone-3-O-rutinoside (rutin).

The use of bisabolol and/or bisabolol oxides as a care-providing agent in the agents according to the present invention is also preferred. Hair treatment agents according to the present invention that additionally include 0.001 to 5 wt %, preferably 0.01 to 4 wt %, particularly preferably 0.02 to 2.5 wt %, and in particular 0.1 to 1.5 wt % bisabolol and/or oxides of bisabolol, preferably (−)-alpha-bisabolol,

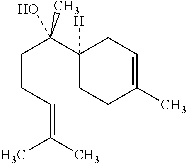 [SMILES: CC(C)=CCC[C@](C)(O)[C@H]1CC=C(C)CC1]

are preferred here.

Creatine (3-methylguanidinoacetic acid) is also suitable according to the present invention as a care-providing substance. Particularly preferred hair treatment agents according to the present invention include, based on their weight, 0.01 to 15 wt %, preferably 0.025 to 12.5 wt %, particularly preferably 0.05 to 10 wt %, more preferably 0.1 to 7.5 wt %, and in particular 0.5 to 5 wt % N-methylguanidinoacetic acid (creatine).

The agents according to the present invention can include, in addition to the ingredients recited above and optional further ingredients, further substances that prevent, mitigate, or cure hair loss. A content of hair-root-stabilizing active substances is particularly advantageous. These substances are described below:

Propecia (finasteride) is at present the only preparation that is approved worldwide and for which effectiveness and compatibility have been demonstrated in numerous studies. The effect of Propecia is that less DHT can form from testosterone.

Minoxidil, with or without supplementary additives, is probably the oldest demonstrably effective hair growth agent. For the treatment of hair loss, it must only be used for external application. Hair lotions exist that include 2% to 5% minoxidil, also gels having up to 15% minoxidil. Effectiveness increases with dosage, but minoxidil is soluble in hair lotions only up to a 5% proportion. In many countries, hair lotions having a minoxidil content of up to 2% are obtainable without a prescription.

To counteract hormonal influences on the hair follicles, spironolactone can be applied for external use in the form of hair lotion and in combination with minoxidil. Spironolactone acts as an androgen receptor blocker, i.e. the binding of DHT to the hair follicles is prevented.

In summary, hair treatment agents according to the present invention that additionally include, based on its weight, 0.001 to 5 wt % hair-root-stabilizing substances, in particular minoxidil and/or finasteride and/or ketoconazole, are preferred.

In addition to the care-providing substances, the agents according to the present invention can include further care-providing substances. Their presence is not obligatorily necessary in order for the effects according to the present invention to be achieved, but farther-reaching effects, such as a pleasant feel or pleasant application haptics, may result from the use of these care-providing substance.

As a first essential ingredient, the agents according to the present invention include 0.1 to 20 wt % of at least one cosmetic oil. These oily substances have a melting point below 50° C., particularly preferably below 45° C., very particularly preferably below 40° C., highly preferably below 35° C., and most preferably the cosmetic oils are still flowable at a temperature below 30° C. Preferred cosmetic oils are defined and described in more detail below.

Included among the natural and synthetic cosmetic oils are, for example:
Vegetable oils. Examples of such oils are sunflower oil, olive oil, soybean oil, rapeseed oil, almond oil, jojoba oil, orange oil, wheat germ oil, peach-kernel oil, and the liquid components of coconut oil. Also suitable, however, are other triglyceride oils such as the liquid components of beef tallow, as well as synthetic triglyceride oils.
Liquid paraffin oils, isoparaffin oils, and synthetic hydrocarbons, as well as di-n-alkyl ethers having a total of between 12 and 36 carbon atoms, in particular 12 to 24 carbon atoms, for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether, and n-hexyl-n-undecyl ether, as well as ditert-butyl ether, diisopentyl ether, di-3-ethyldecyl ether, tert-butyl-n-octyl ether, isopentyl-n-octyl ether, and 2-methylpentyl-n-octyl ether. The compounds 1,3-di-(2-ethylhexyl)cyclohexane (Cetiol® S) and di-n-octyl ether (Cetiol® OE), available as commercial products, can be preferred.
Silicones. These derive preferably from the groups of dimethicones and/or cyclomethicones and/or amodimethicones and/or dimethiconols and/or trisiloxanes.
Ester oils. “Ester oils” are to be understood as esters of C₆ to C₃₀ fatty acids with C₂ to C₃₀ fatty alcohols. The monoesters of fatty acids with alcohols having 2 to 24 carbon atoms are preferred. Examples of fatty-acid components used in the esters are hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof. Examples of the fatty-alcohol components in the ester oils are isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof. Isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred according to the present invention.
dicarboxylic acid esters such as di-n-butyl adipate, di-(2-ethylhexyl) adipate, di-(2-ethylhexyl) succinate, and diisotridecyl acelaate, as well as diol esters such as ethylene glycol dioleate, ethylene glycol diisotridecanoate, propylene glycol di-(2-ethylhexanoate), propylene glycol diisostearate, propylene glycol dipelargonate, butanediol diisostearate, neopentyl glycol dicaprylate,
symmetrical, asymmetrical, or cyclic esters of carbonic acid with fatty alcohols, glycerol carbonate or dicaprylyl carbonate (Cetiol® CC),
fatty acid partial glycerides, i.e. monoglycerides, diglycerides, and industrial mixtures thereof. When industrial products are used, small quantities of triglycerides may still be present for manufacturing-related reasons. The partial glycerides preferably conform to formula (D4-I),

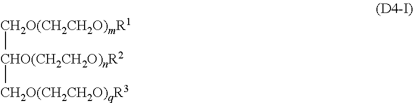

in which R¹, R² and R³ mutually independently denote hydrogen or a linear or branched, saturated and/or unsaturated acyl residue having 6 to 22, preferably 12 to 18 carbon atoms, with the provision that at least one of these groups denotes an acyl residue and at least one of these groups denotes hydrogen. The sum (m+n+q) denotes 0 or numbers from 1 to 100, preferably 0 or 5 to 25. Preferably R¹ denotes an acyl residue and R² and R³ denote hydrogen, and the sum (m+n+q) is 0. Typical examples are mono- and/or diglycerides based on hexanoic acid, octanoic acid, 2-ethylhexanoic acid, decanoic acid, lauric acid, isotridecanoic acid, myristic acid, palmitic acid, palmoleic acid, stearic acid, isostearic acid, oleic acid, elaidic acid, petroselinic acid, linoleic acid, linolenic acid, elaeostearic acid, arachidic acid, gadoleic acid, behenic acid and erucic acid, as well as industrial mixtures thereof. Oleic acid monoglycerides are preferably used.

Preferred cosmetic oils are vegetable oils. Natural oils used are, for example, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn pulp oil, sea buckthorn seed oil, sesame oil, shea butter, soy oil, sunflower oil, grapeseed oil, walnut oil, or wild rose oil.

Preferred natural oils include at least the fatty acids palmitic acid, stearic acid, and linoleic acid. Particularly preferred natural oils include the fatty acids palmitic acid, stearic acid, and linoleic acid in a total quantity of at least 50 wt % of the fatty acids. Very particularly preferred oils are furthermore notable for an additional squalene content. Most preferred natural oils and mixtures thereof also comprise a proportion of linolenic acids.

The teaching of the present invention of course also comprises the fact that at least two natural oils can be mixed with one another. Preferred mixtures of the natural oils are amaranth seed oil with at least one sea buckthorn oil, amaranth seed oil with shea butter, amaranth seed oil with camelina oil, amaranth seed oil with olive oil, amaranth seed oil with macadamia nut oil, olive oil with at least one sea buckthorn oil, olive oil with camelina oil, olive oil with shea butter, macadamia nut oil and at least one sea buckthorn oil, macadamia nut oil with shea butter.

Argan oil is one of the particularly preferred natural oils. A further preferred natural oil is amaranth seed oil. An oil suitable according to the present invention is obtainable, for example, under the commercial name “Amaranth Seed Oil” from the Euro Ingredients company. Shea butter is a further example of the natural oils.

In summary, hair treatment agents preferred according to the present invention are characterized in that they include 0.15 to 15 wt %, preferably 0.2 to 10 wt %, more preferably 0.25 to 7.5 wt %, even more preferably 0.5 to 5 wt %, and in particular 0.75 to 2.5 wt % of at least one vegetable oil from the group of amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, blackcurrant seed oil, jojoba oil, cocoa butter, linseed oil, macadamia nut oil, corn oil, almond oil, marula oil, evening primrose oil, olive oil, palm oil, rapeseed oil, rice oil, sea buckthorn pulp oil, sea buckthorn seed oil, sesame oil, shea butter, soy oil, sunflower oil, grapeseed oil, walnut oil, or wild rose oil.

The ester oils represent a further preferred group of cosmetic oils. Isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are particularly preferred according to the present invention.

In summary, hair treatment agents preferred according to the present invention are characterized in that they include 0.15 to 15 wt %, preferably 0.2 to 10 wt %, more preferably 0.25 to 7.5 wt %, even more preferably 0.5 to 5 wt %, and in particular 0.75 to 2.5 wt % of at least one ester of acids from the group of hexanoic acid, octanoic acid, 2-ethylhexanoic acid, decanoic acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, as well as industrial mixtures thereof with at least one alcohol from the group of isopropyl alcohol, capronyl alcohol, capryl alcohol, 2-ethylhexyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol, and brassidyl alcohol, as well as industrial mixtures thereof, wherein isopropyl myristate, isononanoic acid C16-18 alkyl esters, 2-ethylhexyl palmitate, stearic acid 2-ethylhexyl ester, cetyl oleate, glycerol tricaprylate, coconut fatty alcohol caprinate/caprylate, n-butyl stearate, oleyl erucate, isopropyl palmitate, oleyl oleate, lauric acid hexyl ester, di-n-butyl adipate, myristyl myristate, cetearyl isononanoate, oleic acid decyl ester are preferred.

Further preferred cosmetic oils are symmetrical, asymmetrical, or cyclic esters of carbonic acid with fatty alcohols. Hair treatment agents preferred according to the present invention are characterized here in that they include 0.15 to 15 wt %, preferably 0.2 to 10 wt %, more preferably 0.25 to 7.5 wt %, even more preferably 0.5 to 5 wt %, and in particular 0.75 to 2.5 wt % of at least one ester of carbonic acid from the group of glycerol carbonate and/or dicaprylyl carbonate.

Silicones represent a further preferred group. Silicones derive preferably from the groups of dimethicones and/or cyclomethicones and/or amodimethicones and/or dimethiconols and/or trisiloxanes.

Dimethicones can be both linear and branched, and also cyclic or cyclic and branched. Linear dimethicones can be represented by the following structural formula (Si1):

(SiR¹₃)—O—(SiR²₂—O—)ₓ—(SiR¹₃) (Si1).

Branched dimethicones can be represented by the structural formula (Si1.1):

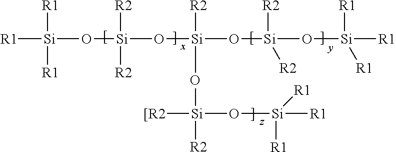

Residues R¹ and R² residues each denote, mutually independently, hydrogen, a methyl residue, a C2 to C30 linear, saturated or unsaturated hydrocarbon residue, a phenyl residue, and/or an aryl residue. Non-limiting examples of the residues represented by R¹ and R² include alkyl residues such as methyl, ethyl, propyl, isopropyl, butyl, isobutyl, pentyl, isopentyl, neopentyl, amyl, isoamyl, hexyl, isohexyl and the like; alkenyl residues such as vinyl, halovinyl, alkylvinyl, allyl, haloallyl, alkylallyl; cycloalkyl residues such as cyclobutyl, cyclopentyl, cyclohexyl, and the like; phenyl residues, benzyl residues, halogenated hydrocarbon residues such as 3-chloropropyl, 4-bromobutyl, 3,3,3-trifluoropropyl, chlorocyclohexyl, bromophenyl, chlorophenyl, and the like, and sulfur-including residues such as mercaptoethyl, mercaptopropyl, mercaptohexyl, mercaptophenyl, and the like; preferably R¹ and R² are an alkyl residue that includes 1 to approximately 6 carbon atoms, and most preferably R¹ and R² are methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂, —CH₂CH(CH₃)C(O)OCH₂—, —(CH₂)₃CC(O)OCH₂CH₂—, —C₆H₄C₆H₄—, —C₆H₄CH₂C₆H₄—, and —(CH₂)₃C(O)SCH₂CH₂—. Methyl, phenyl, and C2 to C22 alkyl residues are preferred as R¹ and R². Among the C2 to C22 alkyl residues, lauryl, stearyl, and behenyl residues are very particularly preferred. The numbers x, y, and z are integers and range, mutually independently in each case, from 0 to 50,000. The molecular weights of the dimethicones are between 1000 D and 10,000,000 D. The viscosities are between 100 and 10,000,000 cPs, measured at 25° C. using a glass capillary viscometer in accordance with Dow Corning Corporate Test Method CTM 0004 of Jul. 20, 1970. Preferred viscosities are between 1000 and 5,000,000 cPs; very particularly preferred viscosities are between 10,000 und 3,000,000 cPs. The most preferred range is between 50,000 und 2,000,000 cPs. Viscosities around the range of approximately 60,000 cPs are most highly preferred. The word “approximately” defines here a deviation, familiar to one skilled in the art of industrially manufactured products, from the value recited after the word “approximately.” Reference may be made here, for example to the product “Dow Corning 200, 60,000 cSt.”

The teaching of the present invention of course also comprises the fact that the dimethicones can already be present as an emulsion.

If the dimethicones are used as an emulsion, the droplet size of the emulsified particles is then, according to the present invention, equal to 0.01 to 10,000 μm, preferably 0.01 to 100 μm, very particularly preferably 0.01 to 20 μm, and most preferably 0.01 to 10 μm. The particle size is determined using the light-scattering method.

Particularly preferred hair treatment agents according to the present invention are characterized in that they include at least one silicone of formula (Si1.2)

(CH₃)₃Si-[O—Si(CH₃)₂]ₓ—O—Si(CH₃)₃ (Si1.2),

in which x denotes a number from 0 to 100, preferably from 0 to 50, more preferably from 0 to 20, and in particular 0 to 10.

Dimethicones (Si1) are included in the compositions according to the present invention preferably in quantities from 0.01 to 10 wt %, preferably 0.01 to 8 wt %, particularly preferably 0.1 to 7.5 wt %, and in particular 0.1 to 5 wt %, based on the total composition.

Particularly preferred agents according to the present invention include one or more aminofunctional silicones. Such silicones can be described, for example, by formula (Si-2)

M(RₐQ_{b}SiO_{(4-a-b)/2})ₓ(R_{c}SiO_{(4-c)/2})_{y}M (Si-2);
in the above formula,
R is a hydrocarbon or a hydrocarbon residue having 1 to approximately 6 carbon atoms,
Q is a polar residue of the general formula —R¹HZ, in which
R¹ is a divalent connecting group that is bound to hydrogen and to the residue Z, assembled from carbon and hydrogen atoms, carbon, hydrogen, and oxygen atoms, or carbon, hydrogen, and nitrogen atoms, and
Z is an organic aminofunctional residue that includes at least one aminofunctional group;
a assumes values in the range from approximately 0 to approximately 2,
b assumes values in the range from approximately 1 to approximately 3,
a+b is less than or equal to 3, and
c is a number in the range from approximately 1 to approximately 3, and
x is a number in the range from 1 to approximately 2,000, preferably from approximately 3 to approximately 50, and most preferably from approximately 3 to approximately 25, and
y is a number in the range from approximately 20 to approximately 10,000, preferably from approximately 125 to approximately 10,000, and most preferably from approximately 150 to approximately 1,000, and
M is a suitable silicone terminal group as known in the existing art, preferably trimethylsiloxy.

Non-limiting examples of the residues represented by R in formula (Si-2) include alkyl residues such as methyl, ethyl, propyl, isopropyl, isopropyl, butyl, isobutyl, amyl, isoamyl, hexyl, isohexyl and the like; alkenyl residues such as vinyl, halovinyl, alkyl vinyl, allyl, haloallyl, cycloalkyl residues such as cyclobutyl, cyclopentyl, cyclohexyl and the like; phenyl residues, benzyl residues, halocarbon residues such as 3-chloropropyl, 4-bromobutyl, 3,3,3-trifluoropropyl, chlorocyclohexyl, bromophenyl, chlorophenyl, and the like, and sulfur-including residues such as mercaptoethyl, mercaptopropyl, mercaptohexyl, mercaptophenyl and the like; R is preferably an alkyl residue that includes 1 to approximately 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—, and —(CH₂)₃C(O)SCH₂CH₂—.

Z according to formula (Si-2) is an organic aminofunctional residue including at least one functional amino group. One possible formula for said Z is NH(CH₂)_{z}NH₂, in which z is an integer greater than or equal to 1. Another possible formula for said Z is —NH(CH₂)_{z}NH(CH₂)_{zz}NH, in which both z and zz, mutually independently, are an integer greater than or equal to 1; this structure encompasses diamino ring structures such as piperazinyl. Said Z is particularly preferably an —NHCH₂CH₂NH₂ residue. Another possible formula for said Z is —N(CH₂)_{z}(CH₂)_{zz}NX₂ or —NX₂, in which each X is selected, independently of X₂, from the group comprising hydrogen and alkyl groups having 1 to 12 carbon atoms, and zz is 0.

Q in formula (Si-2) is most preferably a polar aminofunctional residue of the formula —CH₂CH₂CH₂NHCH₂CH₂NH₂.

In formula (Si-2) “a” assumes values in the range from 0 to 2, “b” assumes values in the range from 2 to 3, “a”+“b” is less than or equal to 3, and “c” is a number in the range from 1 to 3. The molar ratio of the RₐQ_{b}SiO_{(4-a-b)/2} units to the R_{c}SiO_{(4-c)/2} units is in the range from approximately 1:2 to 1:65, preferably from approximately 1:5 to approximately 1:65, and most preferably from approximately 1:15 to approximately 1:20. If one or more silicones of the above formula (Si-2) are used, the different variable substituents in the above formula can then be different in the different silicone components that are present in the silicone mixture.

Preferred hair treatment agents according to the present invention include an aminofunctional silicone of formula (Si-3)

R′ₐG₃₋ₐ-Si(OSiG₂)ₙ—(OSiG_{b}R′_{2-b})ₘ—O—SiG₃₋ₐ-R′ₐ (Si-3),
in which
G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, CH(CH₃)CH₂CH₃, —O—C(CH₃)₃, —C(CH₃)₃;
a denotes a number between 0 and 3, in particular 0;
b denotes a number between 0 and 1, in particular 1,
m and n are numbers whose sum (m+n) is between 1 and 2000, preferably between 50 and 150, n preferably assuming values from 0 to 1999 and in particular from 49 to 149, and m preferably assuming values from 1 to 2000, in particular from 1 to 10;
R′ is a monovalent residue selected from
   -Q-N(R″)—CH₂—CH₂—N(R″)₂,
   -Q-N(R″)₂,
   -Q-N⁺(R″)₃A
   -Q-N⁺H(R″)₂A⁻,
   -Q-N⁺H₂(R″)₂A⁻,
   -Q-N(R″)—CH₂—CH₂—N⁺R″H₂A⁻,
      wherein each Q denotes a chemical bond, —CH₂—, —CH₂—CH₂—, —CH₂CH₂CH₂, —C(CH₃)₂—, —CH₂CH₂CH₂CH₂—, —CH₂C(CH₃)₂—, —CH(CH₃)CH₂CH₂—,
      R″ denotes identical or different residues from the group of —H, phenyl, benzyl, CH₂—CH(CH₃)Ph, the C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion that is preferably selected from chloride, bromide, iodide, or methosulfate.

Cationic silicone oils such as, for example, the commercially obtainable Dow Corning 929 Emulsion (including a hydroxylamino-modified silicone that is also referred to as Amodimethicone), DC 2-2078 (manufacturer: Dow Corning; INCI name: Aminopropyl Phenyl Trimethicone), DC 5-7113 (manufacturer: Dow Corning; INCI name: Silicone Quaternium 16), SM-2059 (manufacturer: General Electric), SLM-55067 (manufacturer: Wacker), as well as Abil®-Quat 3270 and 3272 (manufacturer: Th. Goldschmidt; diquaternary polydimethylsiloxanes, Quaternium-80), are suitable according to the present invention.

Particularly preferred agents according to the present invention are characterized in that they include at least one aminofunctional silicone of formula (Si3-a)

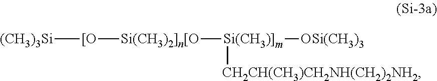

in which m and n are numbers whose sum (m+n) is between 1 and 2000, preferably between 50 and 150, where n assumes values preferably from 0 to 1999 and in particular from 49 to 149, and m assumes values preferably from 1 to 2000, in particular from 1 to 10.

These silicones are referred to according to the INCI declaration as Trimethylsilylamodimethicones and are obtainable, for example, under the designation Q2-7224 (manufacturer: Dow Corning; a stabilized trimethylsilylamodimethicone).

Also particularly preferred are agents according to the present invention that include at least one aminofunctional silicone of formula (Si-3b)

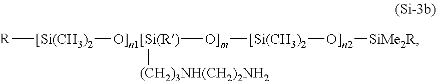

in which
R denotes —OH, an (optionally ethoxylated and/or propoxylated) (C₁ to C₂₀) alkoxy group, or a —CH₃ group,
R′ denotes —OH, a (C₁ to C₂₀) alkoxy group, or a —CH₃ group, and
m, n1, and n2 are numbers whose sum (m+n1+n2) is between 1 and 2000, preferably between 50 and 150, where the sum (n1+n2) assumes values preferably from 0 to 1999 and in particular from 49 to 149, and m assumes values preferably from 1 to 2000, in particular from 1 to 10.

These silicones are referred to according to the INCI declaration as Amodimethicones or as functionalized Amodimethicones, for example Bis(C13-15 Alkoxy) PG Amodimethicone (obtainable e.g. as a commercial product: DC 8500 of the Dow Corning company), Trideceth-9 PG-Amodimethicone (obtainable e.g. as a commercial product: Silcare Silicone SEA of the Clariant company).

Regardless of which aminofunctional silicones are used, cosmetic or dermatological preparations according to the present invention that include an aminofunctional silicone whose amine number is above 0.25 meq/g, preferably above 0.3 meq/g, and in particular above 0.4 meq/g, are preferred. The amine number denotes, in this context, the milliequivalent amine per gram of the aminofunctional silicone. It can be ascertained by titration, and also indicated using the unit of “mg KOH/g”.

Hair treatment agents preferred according to the present invention are characterized in that they include, based on their weight, 0.01 to 10 wt %, preferably 0.1 to 8 wt %, particularly preferably 0.25 to 7.5 wt %, and in particular 0.5 to 5 wt % aminofunctional silicone(s).

The compositions according to the present invention can also include at least one polyammonium-polysiloxane compound as a silicone. Polyammonium-polysiloxane compounds can be acquired, for example, from GE Bayer Silicones under the commercial name Baysilone®. The products having the designations Baysilone TP 3911, SME 253, and SFE 839 are preferred in this context. It is very particularly preferred to use Baysilone TP 3911 as an active component of the compositions according to the present invention.

Polyammonium-polysiloxane compounds are used in the compositions according to the present invention preferably in a quantity from 0.01 to 10 wt %, preferably 0.01 to 7.5, particularly preferably 0.01 to 5.0 wt %, very particularly preferably from 0.05 to 2.5 wt %, referring in each case to the total composition.

The cyclic dimethicones referred to according to INCI as Cyclomethicones are also usable with preference according to the present invention. Preferred here are cosmetic or dermatological preparations according to the present invention that include at least one silicone of formula (Si-4)

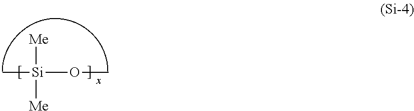

in which x denotes a number from 3 to 200, preferably from 3 to 10, more preferably from 3 to 7, and in particular 3, 4, 5, or 6.

The silicones described above comprise a backbone that is constructed from —Si—O—Si— units. These —Si—O—Si— units can of course also be interrupted by carbon chains. Corresponding molecules are accessible via chain lengthening reactions, and are employed preferably in the form of silicone-in-water emulsions.

Agents likewise preferred according to the present invention are characterized in that they include at least one silicone of formula (Si-5)

R₃Si—[O—SiR₂]ₓ—(CH₂)ₙ[O—SiR₂]_{y}—O—SiR₃ (Si-5),

in which R denotes identical or different residues from the group —II, phenyl, benzyl, —CH₂—CH(CH₃)Ph, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, x and y respectively denote a number from 0 to 200, preferably from 0 to 10, more preferably from 0 to 7, and in particular 0, 1, 2, 3, 4, 5, or 6, and n denotes a number from 0 to 10, preferably from 1 to 8, and in particular 2, 3, 4, 5, 6.

Besides the dimethicones, dimethiconols, amodimethicones, and/or cyclomethicones, water-soluble silicones can be included in the compositions according to the present invention as further silicones.

Corresponding hydrophilic silicones are selected, for example, from compounds of formulas (Si-6) and/or (Si-7). In particular, preferred silicone-based water-soluble silicone-based surfactants are selected from the group of dimethicone copolyols, which are preferably alkoxylated, in particular polyethoxylated or polypropoxylated.

“Dimethicone copolyols” are understood according to the present invention preferably as polyoxyalkylene-modified dimethylpolysiloxanes of the general formulas (Si-6) or (Si-7):

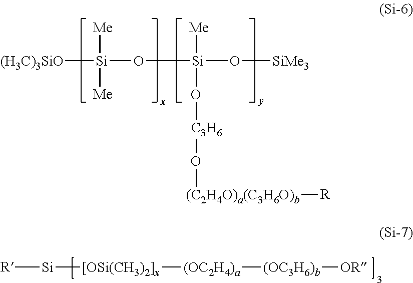

in which
residue R denotes a hydrogen atom, an alkyl group having 1 to 12 carbon atoms, an alkoxy group having 1 to 12 carbon atoms, or a hydroxyl group,
residues R′ and R″ signify alkyl groups having 1 to 12 carbon atoms,
x denotes an integer from 1 to 100, preferably from 20 to 30,
y denotes an integer from 1 to 20, preferably from 2 to 10, and
a and b denote integers from 0 to 50, preferably from 10 to 30.

Particularly preferred dimethicone copolyols for purposes of the invention are, for example, the products marketed commercially under the trade name SILWET (Union Carbide Corporation) and DOW CORNING. Dimethicone copolyols particularly preferred according to the present invention are Dow Corning 190 and Dow Corning 193 (Dow).

Dimethicone copolyols are in the compositions according to the present invention preferably in quantities from 0.01 to 10 wt %, preferably 0.01 to 8 wt %, particularly preferably 0.1 to 7.5 wt %, and in particular 0.1 to 5 wt % dimethicone copolyol based on the composition.

Lastly, dimethiconols (Si8) are understood as silicone compounds. Dimethiconols constitute a further group of silicones that are particularly preferred according to the present invention. Dimethiconols according to the present invention can be both linear and branched, and also cyclic or cyclic and branched. Linear dimethiconols can be represented by the following structural formula (Si8-I):

(SiOHR¹₂)—O—(SiR²₂—O—)ₓ—(SiOHR¹₂) (Si8-I).

Branched dimethiconols can be represented by the structural formula (Si8-II):

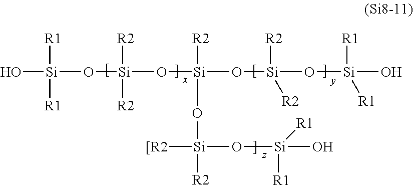

Residues R¹ and R² denote, mutually independently in each case, hydrogen, a methyl residue, a C2 to C30 linear, saturated or unsaturated hydrocarbon residue, a phenyl residue, and/or an aryl residue. Non-limiting examples of the residues represented by R¹ and R² include alkyl residues such as methyl, ethyl, propyl, isopropyl, butyl, isobutyl, pentyl, isopentyl, neopentyl, amyl, isoamyl, hexyl, isohexyl and the like; alkenyl residues such as vinyl, halovinyl, alkyl vinyl, allyl, haloallyl, alkylallyl; cycloalkyl residues such as cyclobutyl, cyclopentyl, cyclohexyl and the like; phenyl residues, benzyl residues, halocarbon residues such as 3-chloropropyl, 4-bromobutyl, 3,3,3-trifluoropropyl, chlorocyclohexyl, bromophenyl, chlorophenyl, and the like, and sulfur-including residues such as mercaptoethyl, mercaptopropyl, mercaptohexyl, mercaptophenyl and the like; R¹ and R² are preferably an alkyl residue that includes 1 to approximately 6 carbon atoms, and R¹ and R² are most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—, and —(CH₂)₃C(O)SCH₂CH₂—. Methyl, phenyl, and C2 to C22 alkyl residues are preferred as R¹ and R². Among the C2 to C22 alkyl residues, lauryl, stearyl, and behenyl residues are very particularly preferred. The numbers x, y, and z are integers and range, mutually independently in each case, from 0 to 50,000. The molecular weights of the dimethiconols are between 1000 D and 10,000,000 D. The viscosities are between 100 and 10,000,000 cPs, measured at 25° C. using a glass capillary viscometer in accordance with Dow Corning Corporate Test Method CTM 0004 of Jul. 20, 1970. Preferred viscosities are between 1000 and 5,000,000 cPs; very particularly preferred viscosities are between 10,000 and 3,000,000 cPs. The most preferred range is between 50,000 and 2,000,000 cPs.

If the dimethiconols according to the present invention are used as an emulsion, the droplet size of the emulsified particles is then, according to the present invention, equal to 0.01 μm to 10,000 μm, preferably 0.01 to 100 μm, very particularly preferably 0.01 to 20 μm, and most preferably 0.01 to 10 μm. The particle size is determined using the light-scattering method.

The following commercial products are recited as examples of such products: Dow Corning 1-1254 Fluid, Dow Corning 2-9023 Fluid, Dow Corning 2-9026 Fluid, X-21-5619 (Shin-Etsu Chemical Co.), Abil OSW 5 (Degussa Care Specialties), ACC DL-9430 Emulsion (Taylor Chemical Company), Dow Corning 1401 Fluid, Dow Corning 1403 Fluid, Dow Corning 1501 Fluid, Dow Corning 1784 HVF Emulsion, Dow Corning 9546 Silicone Elastomer Blend All the aforementioned Dow Corning Corporation), Silsoft 148, Silsoft E-50, Silsoft E-623 (all the aforesaid Crompton Corporation), SM555, SM2725, SM2765, SM2785 (all the aforesaid GE Silicones), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all the aforesaid Wacker-Chemie GmbH).

Dimethiconols (Si8) are in the compositions according to the present invention preferably in quantities from 0.01 to 10 wt %, preferably 0.01 to 8 wt %, particularly preferably 0.1 to 7.5 wt %, and in particular 0.1 to 5 wt % dimethiconol, based on the composition.

If a mixture of at least two silicones is used, that mixture is then included in the compositions according to the present invention in quantities from 0.01 to 10 wt %, preferably 0.01 to 8 wt %, particularly preferably 0.1 to 7.5 wt %, and in particular 0.1 to 5 wt % silicone mixture, based on the composition.

In summary, hair treatment agents preferred according to the present invention are characterized in that they include 0.15 to 15 wt %, preferably 0.2 to 10 wt %, more preferably 0.25 to 7.5 wt %, even more preferably 0.5 to 5 wt %, and in particular 0.75 to 2.5 wt % of at least one silicone from the groups of dimethicones and/or cyclomethicones and/or amodimethicones and/or dimethiconols and/or trisiloxanes.

The agents according to the present invention can, with particular preference, include one or more amino acids as a further ingredient. Amino acids usable particularly preferably according to the present invention derive from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cyss), L-carnitine, L-citrulline, L-theanine, 3′,4′-dihydroxy-L-phenylalanine (L-DOPA), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the present invention include one or more amino acids in narrower quantity ranges. Hair treatment agents preferred according to the present invention are characterized here in that they include as a care-providing substance, based on their weight, 0.01 to 5 wt %, preferably 0.02 to 2.5 wt %, particularly preferably 0.05 to 1.5 wt %, more preferably 0.075 to 1 wt %, and in particular 0.1 to 0.25 wt % amino acid(s), preferably from the group of glycine and/or alanine and/or valine and/or lysine and/or leucine and/or threonine.

As a further constituent, the agents according to the present invention can include at least one carbohydrate from the group of the monosaccharides, disaccharides, and/or oligosaccharides. Hair treatment agents preferred according to the present invention are characterized here in that they include as a care-providing substance, based on their weight, 0.01 to 5 wt %, preferably 0.05 to 4.5 wt %, particularly preferably 0.1 to 4 wt %, more preferably 0.5 to 3.5 wt %, and in particular 0.75 to 2.5 wt % carbohydrate(s) selected from monosaccharides, disaccharides, and/or oligosaccharides, preferred carbohydrates being selected from
monosaccharides, in particular D-ribose and/or D-xylose and/or L-arabinose and/or

D-glucose and/or D-mannose and/or D-galactose and/or D-fructose and/or sorbose and/or L-fucose and/or L-rhamnose,
disaccharides, in particular sucrose and/or maltose and/or lactose and/or trehalose and/or cellobiose and/or gentobiose and/or isomaltose.

Particularly preferred agents according to the present invention include, based on their weight,
0.005 to 0.015 wt % caffeine and 0.75 to 1.5 wt % glucose monohydrate,
0.005 to 0.015 wt % caffeine and 0.75 to 1.5 wt % sucrose,
0.005 to 0.015 wt % caffeine and 0.75 to 1.5 wt % fructose.

As already mentioned, preferred agents according to the present invention include (an) amino acids(s).

Amino acids usable particularly preferably according to the present invention derive from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-cyss), L-carnitine, L-citrulline, L-theanine, 3′,4′-dihydroxy-L-phenylalanine (L-DOPA), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine, L-ornithine, wherein both the individual amino acids and mixtures can be used.

Preferred agents according to the present invention include one or more amino acids in narrower quantity ranges. Cosmetic agents preferred according to the present invention are characterized here in that they additionally include 0.05 to 5 wt %, preferably 0.1 to 2.5 wt %, particularly preferably 0.15 to 1 wt %, and in particular 0.2 to 0.5 wt % amino acid(s), preferably (an) amino acid(s) from the group of glycine and/or alanine and/or valine and/or lysine and/or leucine and/or threonine.

Surfactants represent a particularly preferred group of ingredients.

The concentration of surfactants from the individual groups can vary depending on the intended application; anionic surfactant(s) are particularly preferred in cleaning formulations (in particular in shampoos).

All anionic surface-active substances suitable for use on the human body are suitable as anionic surfactants and emulsifier agents for the compositions according to the present invention. These substances are characterized by an anionic group imparting water solubility, for example a carboxylate, sulfate, sulfonate, or phosphate group, and a lipophilic alkyl group having approximately 8 to 30 carbon atoms. Glycol ether or polyglycol ether groups, ester, ether, and amide groups, and hydroxyl groups can additionally be included in the molecule. Examples of suitable anionic surfactants and emulsifiers are, in each case in the form of the sodium, potassium, and ammonium and mono-, di, and trialkanolammonium salts having 2 to 4 carbon atoms in the alkanol group:
linear and branched fatty acids having 8 to 30 carbon atoms (soaps);
ethercarboxylic acids of the formula R—O—(CH₂—CH₂O)ₓ—CH₂—COOH, in which R is a linear alkyl group having 8 to 30 carbon atoms and x=0 or is 1 to 16;
acyl sarcosides having 8 to 24 carbon atoms in the acyl group;
acyl taurides having 8 to 24 carbon atoms in the acyl group;
acyl isethionates having 8 to 24 carbon atoms in the acyl group;
linear alkanesulfonates having 8 to 24 carbon atoms;
linear alpha-olefinsulfonates having 8 to 24 carbon atoms;
alpha-sulfo fatty acid methyl esters of fatty acids having 8 to 30 carbon atoms;
acyl glutamates of formula (I)

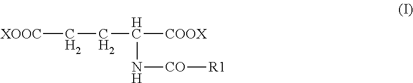

in which R¹CO denotes a linear or branched acyl residue having 6 to 22 carbon atoms and 0, 1, 2, or 3 double bonds, and X denotes hydrogen, an alkali metal or alkaline earth metal, ammonium, alkylamtnonium, alkanolammonium, or glucammonium, for example acyl glutamates that derive from fatty acids having 6 to 22, preferably 12 to 18 carbon atoms, such as e.g. C_{12/14} or C_{12/18} coconut fatty acids, lauric acid, myristic acid, palmitic acid, and/or stearic acid, in particular sodium-N-cocoyl-L-glutamate and sodium-N-stearoyl-L-glutamate;
esters of a hydroxy-substituted di- or tricarboxylic acid of the general formula (II):

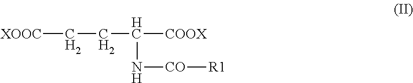

in which X=H or a —CH₂COOR group, Y=H or is —OH, with the condition that Y=H if X=CH₂COOR, R, R¹, and R² mutually independently signify a hydrogen atom, an alkali-metal or alkaline-earth-metal cation, an ammonium group, the cation of an organoammonium base, or a residue Z that derives from a polyhydroxylated organic compound that are selected from the group of etherified (C₆ to C₁₈) alkylpolysaccharides having 1 to 6 monomeric saccharide units and/or etherified aliphatic (C₆ to C₁₆) hydroxyalkyl polyols having 2 to 16 hydroxyl residues, with the provision that at least one of the groups R, R¹, or R² is a residue Z,
esters of sulfosuccinic acid or of sulfosuccinates, of the general formula (III)

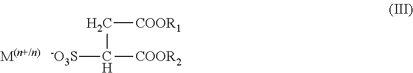

in which M^{(n+/n)}, for n=1, denotes a hydrogen atom, an alkali-metal cation, an ammonium group, or the cation of an organoammonium base, and for n=2, represents an alkaline-earth-metal cation, and R¹ and R² mutually independently denote a hydrogen atom, an alkali-metal or alkaline-earth-metal cation, an ammonium group, the cation of an organoammonium base, or a residue Z that derives from a polyhydroxylated organic compound that is selected from the group of etherified (C₆ to C₁₈) alkylpolysaccharides having 1 to 6 monomeric saccharide units and/or etherified aliphatic (C₆ to C₁₆) hydroxyalkyl polyols having 2 to 16 hydroxyl residues, with the provision that at least one of the groups R, R¹, or R² is a residue Z;
sulfosuccinic acid mono- and dialkyl esters having 8 to 24 carbon atoms in the alkyl group, and sulfosuccinic acid monoalkylpolyoxyethyl esters having 8 to 24 carbon atoms in the alkyl group and 1 to 6 oxyethyl groups;
alkyl sulfates and alkyl polyglycol ether sulfates of the formula R—(O—CH₂—CH₂)x-OSO₃H, in which R is a preferably linear alkyl group having 8 to 30 carbon atoms, and x=0 or is 1 to 12;
mixed surface-active hydroxysulfonates according to DE-A-37 25 030;
esters of tartaric acid and citric acid with alcohols that represent addition products of approximately 2 to 15 molecules of ethylene oxide and/or propylene oxide with C₈₋₂₂ fatty alcohols;
alkyl and/or alkenyl ether phosphates;
sulfated fatty acid alkylene glycol esters;
monoglyceride sulfates and monoglyceride ether sulfates.

Preferred anionic surfactants are acyl glutamates, acyl isethionates, acyl sarcosinates, and acyl taurates, in each case having a linear or branched acyl residue having 6 to 22 carbon atoms and 0, 1, 2, or 3 double bonds, which in particularly preferred embodiments is selected from an octanoyl, decanoyl, lauroyl, myristoyl, palmitoyl, and stearoyl residue, esters of tartaric acid, citric acid, or succinic acid, or the salts of these acids with alkylated glucose, in particular the products having the INCI names Disodium Coco-Glucoside Citrate, Sodium Coco-Glucoside Tartrate, Disodium Coco-Glucoside Sulfosuccinate, alkyl polyglycol ether sulfates and ethercarboxylic acids having 8 to 18 carbon atoms in the alkyl group and up to 12 ethoxy groups in the molecule, sulfosuccinic acid mono- and dialkyl esters having 8 to 18 carbon atoms in the alkyl group, and sulfosuccinic acid monoalkylpolyoxyethyl esters having 8 to 18 carbon atoms in the alkyl group and 1 to 6 ethoxy groups.

Regardless of the type of anionic surfactants used, hair treatment agents according to the present invention that include, based on their weight, 2.5 to 35 wt %, preferably 5 to 30 wt %, more preferably 7.5 to 27.5 wt %, even more preferably 10 to 25 wt %, and in particular 12.5 to 22.5 wt % anionic surfactant(s) are preferred.

Very particularly preferred agents according to the present invention include fatty alcohol sulfates and/or fatty alcohol ether sulfates. Hair treatment agents according to the present invention which are characterized in that they include as an anionic surfactant, based on their weight, 0.1 to 20 wt %, preferably 0.25 to 17.5 wt %, and in particular 2 to 15 wt % fatty alcohol sulfates of the formula

H₃C—(CH₂)ₙ—OSO₃⁻M⁺

in which n denotes values from 5 to 21, preferably from 7 to 19, particularly preferably from 9 to 17, and in particular from 11 to 13, and M denotes a cation from the group Na⁺, K⁺, NH₄⁺, ½ Mg²⁺, ½ Zn²⁺, preferably Na⁺, are therefore preferred embodiments of the present invention.

Further preferred hair treatment agents according to the present invention are characterized in that they include as an anionic surfactant, based on their weight, 0.1 to 20 wt %, preferably 0.25 to 17.5 wt %, and in particular 2 to 15 wt % fatty alcohol ether sulfates of the formula

H₃C—(CH₂)ₙ—(OCH₂CH₂)ₖ—OSO₃⁻M⁺

in which n denotes values from 5 to 21, preferably from 7 to 19, particularly preferably from 9 to 17, and in particular from 11 to 13, and k denotes values of 1, 2, 3, 4, 5, 6, 7, 8, 9, or 10, preferably 1, 2, or 3, and in particular 2, and M denotes a cation from the group Na⁺, K⁺, ½ Mg²⁺, ½ Zn²⁺, preferably Na⁺.

The agents according to the present invention can include further surfactants in addition to the anionic surfactants.

With particular preference, the agents according to the present invention include amphoteric surfactant(s). “Ampholytic” surfactants and emulsifier agents are understood to be those surface-active compounds which include, besides a C₈ to C₂₄ alkyl or acyl group, at least one free amino group and at least one —COOH or —SO₃H group, and are capable of forming internal salts. Examples of suitable ampholytic surfactants are N-alkylglycines, N-alkylaminopropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, having in each case approximately 8 to 24 carbon atoms in the alkyl group. Particularly preferred ampholytic surfactants are N-cocalkylaminopropionate, cocacylaminoethylaminopropionate, and C₁₂ to C₁₈ acyl sarcosine.

Particularly preferred hair treatment agents according to the present invention are characterized in that they include amphoteric surfactant(s) from the groups of
N-alkylglycines,
N-alkylpropionic acids,
N-alkylaminobutyric acids,
N-alkyliminodipropionic acids,
N-hydroxyethyl-N-alkylamidopropyl glycines,
N-alkyltaurines,
N-alkylsarcosines,
2-alkylaminopropionic acids each having approximately 8 to 24 carbon atoms in the alkyl group,
alkylaminoacetic acids each having approximately 8 to 24 carbon atoms in the alkyl group,
N-cocalkylaminopropionate,
cocacylaminoethylaminopropionate,
C₁₂ to C₁₈ acyl sarcosine,
N-alkyl-N,N-dimethylammonium glycinates, for example cocalkyldimethylammonium glycinate,
N-acylaminopropyl-N,N-dimethylammonium glycinates, for example cocacylaminopropyldimethylammonium glycinate,
2-alkyl-3-carboxymethyl-3-hydroxyethylimidazolines each having 8 to 18 carbon atoms in the alkyl or acyl group,
cocacylaminoethylhydroxyethylcarboxymethyl glycinate,
the compounds known by the INCI name Cocamidopropyl Betaine,
the compounds known by the INCI name Disodium Cocamphodiacetate,
   wherein preferred agents include the amphoteric surfactant(s) in quantities from 0.5 to 9 wt %, preferably from 0.75 to 8 wt %, and in particular from 1 to 7.5 wt %, based in each case on the total agent.

Particularly preferred hair treatment agents include, as amphoteric surfactants, betaines of formula (Bet-I)

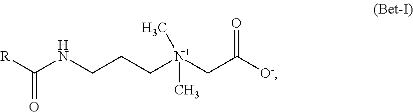

in which R denotes a straight-chain or branched, saturated or mono- or polyunsaturated alkyl or alkenyl residue having 8 to 24 carbon atoms.

These surfactants are referred to according to INCI nomenclature as Amidopropyl Betaines; the representatives that derive from coconut fatty acids are preferred, and are referred to as Cocamidopropyl Betaines. It is particularly preferred according to the present invention to use surfactants of formula (Bet-I) that are a mixture of the following representatives:

H₃C—(CH₂)₇—C(O)—NH—(CH₂)₃N⁺(CH₃)₂CH₂COO⁻

H₃C—(CH₂)₉—C(O)—NH—(CH₂)₃N⁺(CH₃)₂CH₂COO⁻

H₃C—(CH₂)₁₁—C(O)—NH—(CH₂)₃N⁺(CH₃)₂CH₂COO⁻

H₃C—(CH₂)₁₃—C(O)—NH—(CH₂)₃N⁺(CH₃)₂CH₂COO⁻

H₃C—(CH₂)₁₅—C(O)—NH—(CH₂)₃N⁺(CH₃)₂CH₂COO⁻

H₃C—(CH₂)₇—CH═CH—(CH₂)₇—C(O)—NH—(CH₂)₃N⁺(CH₃)₂CH₂COO⁻.

Particularly preferably, surfactants of formula (Bet-I) are used within narrower quantity ranges. Agents according to the present invention that include, based on their weight, 0.25 to 8 wt %, more preferably 0.5 to 7 wt %, more preferably 0.75 to 6.5 wt %, and in particular 1 to 5.5 wt % surfactant(s) of formula (Bet-1) are preferred.

In addition to or instead of the amphoteric surfactant of formula (Bet-I), the hair treatment agents according to the present invention can with particular preference include, as amphoteric surfactants, betaines of formula (Bet-II)

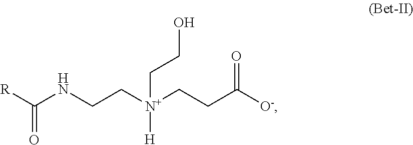

in which R denotes a straight-chain or branched, saturated or mono- or polyunsaturated alkyl or alkenyl residue having 8 to 24 carbon atoms.

These surfactants are referred to according to INCI nomenclature as Amphoacetates, the representatives derived from coconut fatty acids being preferred and being referred to as Cocoamphoacetates.

For production-engineering reasons, surfactants of this type also always include betaines of formula (Bet-IIa)

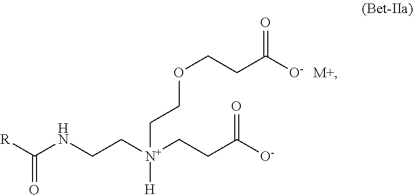

in which R denotes a straight-chain or branched, saturated or mono- or polyunsaturated alkyl or alkenyl residue having 8 to 24 carbon atoms, and M denotes a cation.

These surfactants are referred to according to INCI nomenclature as Amphodiacetates, the representatives derived from coconut fatty acids being preferred and being referred to as Cocoamphodiacetates.

It is particularly preferred to use according to the present invention surfactants of formula (Bet-II) that are a mixture of the following representatives:

H₃C—(CH₂)₇—C(O)—NH—(CH₂)₂NH⁺(CH₂CH₂OH)CH₂CH₂COO⁻;

H₃C—(CH₂)₉—C(O)—NH—(CH₂)₂NH⁺(CH₂CH₂OH)CH₂CH₂COO⁻,

H_{3C}—(CH₂)₁₁—C(O)—NH—(CH₂)₂NH⁺(CH₂CH₂OH)CH₂CH₂COO⁻,

H₃C —(CH₂)₁₃—C(O)—NH—(CH₂)₂NH⁺(CH₂CH₂OH)CH₂CH₂COO⁻,

H₃C—(CH₂)₁₅—C(O)—NH—(CH₂)₂NH⁺(CH₂CH₂OH)CH₂CH₂COO⁻,

H₃C—(CH₂)₇—CH═CH—(CH₂)₇—C(O)—NH—(CH₂)₂NH⁺(CH₂CH₂OH)CH₂CH₂COO⁻.

Surfactants of formula (Bet-II) are used particularly preferably within narrower quantity ranges. Agents according to the present invention that include, based on their weight, 0.25 to 8 wt %, more preferably 0.5 to 7 wt %, more preferably 0.75 to 6.5 wt %, and in particular 1 to 5.5 wt % surfactant(s) of formula (Bet-II) are preferred here.

Hair treatment agents particularly preferred according to the present invention are characterized in that they include 1 to 30 wt %, preferably 1.5 to 25 wt %, more preferably 2 to 20 wt %, even more preferably 2.5 to 15 wt %, and in particular 3 to 10 wt % amphoteric surfactant(s).

In addition to or instead of the amphoteric surfactants, the agents according to the present invention can also include nonionic surfactants.

Particularly preferred nonionic surfactants are alkylpolyglycosides. Alkylpolyglycosides (APG) are nonionic surfactants that are manufactured entirely from renewable raw materials (sugar modules, principally glucose, for example, from corn starch and fatty alcohol, for example, from coconut oil). Alkylpolyglycosides are accessible via an acid-catalyzed reaction (Fischer reaction) from sugars, in particular glucose (or starch) or from butylglycosides with fatty alcohols.

This results in complex mixtures of alkylmonoglucoside (alkyl-α-D- and -β-D-glucopyranoside as well as small proportions of -glucofuranoside), alkyldiglucosides (-isomaltosides, -maltosides, etc.), and alkyloligoglucosides (-maltotriosides, -tetraosides, etc.). The average degree of polymerization of commercial products, whose alkyl residues are in the range from C8 to C16, is 1.2 to 1.5.

It is preferred according to the present invention to use alkylpolyglycosides corresponding to the general formula RO—(Z)ₓ, where R denotes alkyl, Z denotes sugar, and x denotes the number of sugar units.

Those alkylpolyglycosides in which R is made up
substantially of C₈ and Cm alkyl groups,
substantially of C₁₂ and C₁₄ alkyl groups,
substantially of C₈ to C₁₆ alkyl groups, or
substantially of C₁₂ to C₁₆ alkyl groups, or
substantially of C₁₆ to C₁₈ alkyl groups
   are particularly preferred.

Any mono- or oligosaccharides can be used as sugar module Z. Sugars having 5 or 6 carbon atoms, as well as the corresponding oligosaccharides, are usually used. Such sugars are, for example, glucose, fructose, galactose, arabinose, ribose, xylose, lyxose, allose, altrose, mannose, gulose, idose, talose, and sucrose. Preferred sugar modules are glucose, fructose, galactose, arabinose, and sucrose. Preferred hair treatment agents according to the present invention are therefore characterized in that they include, based on their weight, 0.1 to 20 wt %, preferably 1 to 10 wt %, and in particular 2 to 8 wt % alkylpolyglycoside(s) of the formula

H₃C—(CH₂)ₙ—O—(Z)ₓ,

in which n denotes values from 5 to 21, preferably from 7 to 19, particularly preferably from 9 to 17, and in particular from 11 to 13, and k denotes values from 1.1 to 1.8, preferably from 1.2 to 1.5, and Z denotes a sugar module from the group of glucose, fructose, galactose, arabinose, ribose, xylose, lyxose, allose, altrose, mannose, gulose, idose, talose, and sucrose.

Glucose is a particularly preferred sugar module (Z), so that preferred hair treatment agents according to the present invention are characterized in that they include, based on their weight, 0.1 to 15 wt %, preferably 1 to 10 wt %, and in particular 2 to 8 wt % alkylpolyglucoside(s) of the formula

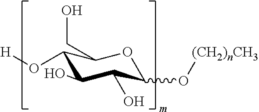

in which n denotes values from 5 to 21, preferably from 7 to 19, particularly preferably from 9 to 17, and in particular from 11 to 13, and m denotes values from 1.1 to 1.8, preferably from 1.2 to 1.5.

The alkylpolyglycosides usable according to the present invention include on average 1.1 to 5 sugar units. Alkylpolyglycosides having values of x from 1.1 to 2.0 are preferred. Alkylglycosides in which x is equal to 1.1 to 1.8 are very particularly preferred.

Very particularly preferred alkylpolyglucosides are those whose alkyl residue is a lauryl residue. In the context of substance mixtures from natural sources, those sources that have a high proportion of C12 fatty acids, in particular coconut fatty acids, are preferred. Particularly preferred hair treatment agents according to the present invention are therefore characterized in that they include, based on their weight, 0.1 to 15 wt %, preferably 1 to 10 wt %, and in particular 2 to 8 wt % alkylpolyglucoside(s) in which n denotes the value 11 and m denotes values from 1.1 to 1.8, preferably from 1.2 to 1.5.

Cationic quaternary compounds can furthermore be used with advantage in the compositions according to the present invention. Quaternary ammonium compounds are, in principle, monomeric cationic or amphoteric ammonium compounds, monomeric amines, aminoamides, polymeric cationic ammonium compounds, and polymeric amphoteric ammonium compounds. From this plurality of possible quaternary ammonium compounds, the groups below have proven to be particularly suitable and are used, considered individually in each case, in a quantity form 0.1 to 10.0 wt %. The quantity does not fall below or exceed this value even if a mixture of different compounds of the quaternary ammonium compounds is used.

Esterquats according to formula (Tkat1-2) constitute the first group. It is the case here that the esterquats must be structurally different from and not identical to the esterquats according to claim 1.

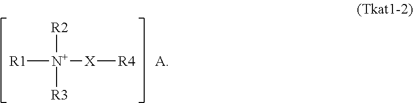

Residues R1, R2, and R3 herein are each mutually independent and can be identical or different. The residues R1, R2, and R3 signify:
a branched or unbranched alkyl residue having 1 to 4 carbon atoms, which can include at least one hydroxyl group, or
a saturated or unsaturated, branched or unbranched, or a cyclic saturated or unsaturated alkyl residue having 6 to 30 carbon atoms, which can include at least one hydroxyl group, or
an aryl or alkaryl residue, for example phenyl or benzyl,
the residue (—X—R4), provided that at most two of the residues R1, R2, or R3 can denote this residue.

The residue (X—R4) is included at least one to three times.

In this, X denotes:
1) —(CH2)n-, where n=1 to 20, preferably n=1 to 10, and particularly preferably n=1 to 5, or
2) —(CH2-CHR5-O)n-, where n=1 to 200, preferably 1 to 100, particularly preferably 1 to 50, and particularly preferably 1 to 20, where R5 has the meaning of hydrogen, methyl, or ethyl,
3) a hydroxyalkyl group having one to four carbon atoms, which can be branched or unbranched and which includes at least one and at most 3 hydroxy groups. Examples are: CHOH, —CHCH₂OH, —CH₂CHOH, —COHCHOH, —CHOHCOH, —CHCHOHCH₃, —CH₂COHCH₃, —CH₂CHOHCH₂—, —C(CH₂OH)₂, —CH₂CHOHCH₂OH, —CH₂CH₂CHOH, —CH₂COHCH₃, and hydroxybutyl residues, the bond from —X— to R4 proceeding from the free valence of the relevant carbon atom,
   and R4 denotes:
2) R6-O—CO—, in which R6 is a saturated or unsaturated, branched or unbranched, or a cyclic saturated or unsaturated alkyl residue having 6 to 30 carbon atoms, which can include at least one hydroxy group, and which optionally can be further oxyethylated with 1 to 100 ethylene oxide units and/or 1 to 100 propylene oxide units, or
3) R7-CO, in which R7 is a saturated or unsaturated, branched or unbranched, or a cyclic saturated or unsaturated alkyl residue having 6 to 30 carbon atoms, which can include at least one hydroxy group, and which optionally can be further oxyethylated with 1 to 100 ethylene oxide units and/or 1 to 100 propylene oxide units,
   and A denotes a physiologically acceptable organic or inorganic anion and is defined at this juncture representatively for all structures including those described hereinafter. The anion of all cationic compounds described is selected from the halide ions fluoride, chloride, bromide, iodide, sulfates of the general formula RSO₃⁻, in which R has the meaning of a saturated or unsaturated alkyl residue having 1 to 4 carbon atoms, or anionic residues of organic acids such as maleate, fumarate, oxalate, tartrate, citrate, lactate, or acetate.

Such products are marketed, for example, under the trademarks Rewoquat®, Stepantex®, Dehyquart®, Armocare®, and Akypoquat®. The products Armocare® VGH-70, Dehyquart® F-75, Dehyquart® C-4046, Dehyquart® L80, Dehyquart® F-30, Dehyquart® AU-35, Rewoquat® WE18, Rewoquat® WE38 DPG, Stepantex® VS 90, and Akypoquat® 131 are examples of these esterquats.

Further compounds of formula (Tkat1-2) that are particularly preferred according to the present invention conform to formula (Tkat1-2.1), the cationic betaine esters

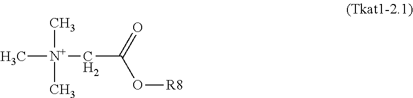

The meaning of R8 corresponds to that of R7.

The esterquats having the commercial names Armocare® VGH-70 as well as Dehyquart® F-75, Dehyquart® L80, Stepantex® VS 90, and Akypoquat® 131 are particularly preferred.

Quaternary imidazoline compounds are a further group. Formula (Tkat2) presented below shows the structure of these compounds.

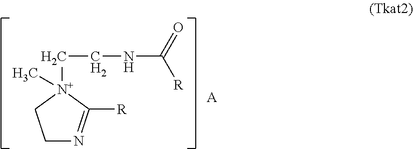

Residues R denote, mutually independently in each case, a saturated or unsaturated, linear or branched hydrocarbon residue having a chain length from 8 to 30 carbon atoms. The preferred compounds of formula (Tkat2) each include the same hydrocarbon residue for R. The chain length of residues R is preferably 12 to 21 carbon atoms. “A” denotes an anion as described above. Examples that are particularly in accordance with the present invention are obtainable, for example, under the INCI names Quaternium-27, Quaternium-72, Quaternium-83, and Quaternium-91. Quaternium-91 is most preferred according to the present invention.

In a particularly preferred embodiment of the invention the agents according to the present invention furthermore include at least one amine and/or cationized amine, in particular an amidoamine and/or a cationized amidoamine, having the following structural formulas:

R1-NH—(CH₂)n-N⁺R²R³R⁴A (Tkat3),

in which R1 signifies an acyl or alkyl residue having 6 to 30 carbon atoms which can be branched or unbranched, saturated or unsaturated, and where the acyl residue and/or the alkyl residue can include at least one OH group, and R2, R3, and R4, mutually independently in each case, signify
1) hydrogen, or
2) an alkyl residue having 1 to 4 carbon atoms, which can be identical or different, saturated or unsaturated, and
3) a branched or unbranched hydroxyalkyl group having one to 4 carbon atoms, having at least one and at most three hydroxy groups, for example —CH₂OH, —CH₂CH₂OH, —CHOHCHOH, —CH₂CHOHCH₃, —CH(CH₂OH)₂, —COH(CH₂OH)₂, —CH₂CHOHCH₂OH, —CH₂CH₂CH₂OH, and hydroxybutyl residues, and
   A signifies an anion as described above, and
   n signifies an integer between 1 and 10.

A composition in which the amine and/or the quaternized amine according to the general formulas (Tkat3) is an amidoamine and/or a quaternized amidoamine, in which R1 signifies a branched or unbranched, saturated or unsaturated acyl residue having 6 to 30 carbon atoms, which can include at least one OH group, is preferred. A fatty acid residue made of oils and waxes, in particular natural oils and waxes, is preferred here. Suitable examples thereof are lanolin, beeswax, or candelilla wax.

Also preferred are those amidoamines and/or quaternized amidoamines in which R2, R3, and/or R4 in formula (Tkat3) signify a residue according to the general formula CH₂CH₂OR5, in which R5 can have the meaning of alkyl residues having 1 to 4 carbon atoms, hydroxyethyl, or hydrogen. The preferred value of n in the general formula (Tkat8) is an integer between 2 and 5.

The alkylamidoamines both can be present as such, and can be converted by protonation in a correspondingly acidic solution into a quaternary compound in the composition. The cationic alkylamidoamines are preferred according to the present invention.

Examples of commercial products of this kind according to the present invention are Witcamine® 100, Incromine® BB, Mackine® 401 and other Mackine® grades, Adogen® S 18V and, as permanently cationic aminoamines: Rewoquat® RTM 50, Empigen® CSC, Swanol® Lanoquat DES-50, Rewoquat® UTM 50, Schercoquat® BAS, Lexquat® AMG-BEO, or Incroquat® Behenyl HE.

The cationic surfactants recited above can be used individually or in any desired combinations with one another, quantities between 0.01 and 10 wt %, preferably quantities from 0.01 to 7.5 wt %, and very particularly preferably quantities from 0.1 to 5.0 wt % being included. The best results of all are obtained with quantities from 0.1 to 3.0 wt %, based in each case on the total composition of the respective agent.

Cationic and amphoteric polymers are further quaternary ammonium compounds.

The cationic and/or amphoteric polymers can be homo- or copolymers or polymers based on natural polymers, the quaternary nitrogen groups being included either in the polymer chain or preferably as a substituent on one or more of the monomers. The ammonium-group-including monomers can be copolymerized with non-cationic monomers. Suitable cationic monomers are unsaturated, radically polymerizable compounds that carry at least one cationic group, in particular ammonium-substituted vinyl monomers such as, for example, trialkylmethacryloxyalkylammonium, trialkylacryloxyalkylammonium, dialkyldiallylammonium, and quaternary vinylammonium monomers having cyclic groups including cationic nitrogens, such as pyridinium, imidazolium, or quaternary pyrrolidones, e.g. alkylvinylimidazolium, alkylvinylpyridinium, or alkyvinylpyrrolidone salts. The alkyl groups of these monomers are preferably lower alkyl groups such as, for example, C1 to C7 alkyl groups, particularly preferably C1 to C3 alkyl groups.

The ammonium-group-including monomers can be copolymerized with non-cationic monomers. Suitable comonomers are, for example, acrylamide, methacrylamide; alkyl and dialkyl acrylamide, alkyl and dialkyl methacrylamide, alkyl acrylate, alkyl methacrylate, vinylcaprolactone, vinylcaprolactam, vinylpyrrolidone, vinyl esters, e.g. vinyl acetate, vinyl alcohol, propylene glycol, or ethylene glycol, the alkyl groups of these monomers being preferably C1 to C7 alkyl groups, particularly preferably C1 to C3 alkyl groups.

From the plurality of these polymers, the following have proven to be particularly effective constituents of the active agent complex according to the present invention: homopolymers of the general formula —{CH₂-[CR¹COO—(CH₂)ₘN⁺R²R³R⁴]}ₙX⁻, in which R¹=—H or —CH₃, R², R³, and R⁴ are selected mutually independently from C1 to 4 alkyl, alkenyl, or hydroxyalkyl groups, m=1, 2, 3, or 4, n is a natural number, and X″ is a physiologically acceptable organic or inorganic anion. In the context of these polymers, the ones preferred according to the present invention are those for which at least one of the following conditions is valid: R¹ denotes a methyl group, R², R³, and R⁴ denote methyl groups, m has the value of 2.

Halide ions, sulfate ions, phosphate ions, methosulfate ions, as well as organic ions such as lactate, citrate, tartrate, and acetate ions are appropriate, for example, as physiologically acceptable counter ions X⁻. Methosulfate and halide ions, in particular chloride, are preferred.

An amphoteric polymer that is highly preferred according to the present invention is a copolymer whose composition is as follows: 0.1 to 50% (based on the total number of monomers in the copolymer) monomers of formula (I)

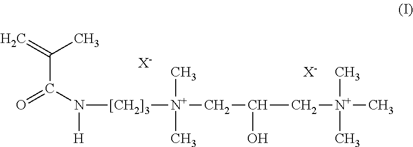

in which X denotes chloride, sulfate, methosulfate,
and monomers A2) from the group of acrylic acid, methacrylic acid, and the alkali metal and ammonium salts of said acids,
the monomers A2) constituting 50 to 99.9%, preferably 50 to 90% (based on the total number of monomers in the copolymer) of the copolymer.

A highly preferred polymer that is constructed as presented above is obtainable commercially under the name Polyquaternium-74.

A particularly suitable homopolymer is the poly(methacryloyloxyethyltrimethylammonium) chloride (crosslinked, if desired) having the INCI name Polyquaternium-37. Such products are available commercially, for example, under the designations Rheocare® CTH (Cosmetic Rheologies) and Synthalen® CR (3V Sigma).

The homopolymer is used preferably in the form of a nonaqueous polymer dispersion. Polymer dispersions of this kind are obtainable commercially under the names Salcare® SC 95 and Salcare® SC 96.

Suitable cationic polymers that are derived from natural polymers are cationic derivatives of polysaccharides, for example cationic derivatives of cellulose, starch, or guar. Chitosan and chitosan derivatives are also suitable. Cationic polysaccharides have the general formula

G-O—B—N+RₐR_{b}R_{c}A⁻

G is an anhydroglucose residue, for example starch anhydroglucose or cellulose anhydroglucose;
B is a divalent connecting group, for example alkylene, oxyalkylene, polyoxyalkylene, or hydroxyalkylene;
Rₐ, R_{b} and R_{c} mutually independently are alkyl, aryl, alkylaryl, arylalkyl, alkoxyalkyl, or alkoxyaryl each having up to 18 carbon atoms, the total number of carbon atoms in Rₐ, R_{b}, and R_{c} preferably being a maximum of 20;
A⁻ is a usual counter anion and is preferably chloride.

Cationic (i.e. quaternized) celluloses are obtainable on the market with different degrees of substitution, cationic charge densities, nitrogen contents, and molecular weights. For example, Polyquaternium-67 is offered commercially under the names Polymer® SL or Polymer® SK (Amerchol). A further highly preferred cellulose is offered by the Croda company under the commercial name Mirustyle® CP. This is a trimonium and cocodimonium hydroxyethyl cellulose, constituting a derivatized cellulose, having the INCI-name Polyquaternium-72. Polyquaternium-72 can be used both in solid form and already predisssolved in aqueous solution.

Further cationic celluloses go by the names Polymer JR® 400 (Amerchol, INCI name Polyquaternium-10) and Polymer Quatrisoft® LM-200 (Amerchol, INCI name Polyquaternium-24). Further commercial products are the compounds Celquat® H 100 and Celquat® L 200. Lastly, a further derivatized cellulose with Trimonium and Cocodimonium Hydroxyethylcellulose, having the INCI name Polyquaternium-72, exists under the commercial name Mirustyle® CP of the Croda company. Polyquaternium-72 can be used both in solid form and already predissolved in aqueous solution. Particularly preferred cationic celluloses are Polyquaternium-10, Polyquaternium-24, Polyquaternium-67, and Polyquaternium-72.

Suitable cationic guar derivatives are marketed under the commercial designation Jaguar® and have the INCI name Guar Hydroxypropyltrimonium Chloride. Particularly suitable cationic guar derivatives are additionally available commercially from the Hercules company under the designation N-Hance. Further cationic guar derivatives are marketed by the Cognis company under the designation Cosmedia®. A preferred cationic guar derivative is the commercial product AquaCat® of the Hercules company. This raw material is a cationic guar derivative that is already predissolved. The cationic guar derivatives are preferred according to the present invention.

A suitable chitosan is marketed, for example, by the Kyowa Oil & Fat company, Japan, under the trade name Flonac. A preferred chitosan salt is chitosonium pyrrolidonecarboxylate, which is marketed e.g. under the designation Kytamer® PC by the Amerchol company, USA. Further chitosan derivatives are readily available commercially under the commercial designations Hydagen® CMF, Hydagen® HCMF, and Chitolam® NB/101.

Further preferred cationic polymers are, for example:
cationized alkylpolyglycosides,
cationized honey, for example the commercial product Honeyquat® 50,
polymeric dimethyldiallylammonium salts and copolymers thereof with esters and amides of acrylic acid and methacrylic acid. The products obtainable commercially under the designations Merquat® 100 (poly(dimethyldiallylammonium) chloride) and Merquat® 550 (dimethyldiallylammonium chloride/acrylamide copolymer) are examples of such cationic polymers, having the INCI name Polyquaternium-7,
vinylpyrrolidone/vinylimidazolium methochloride copolymers, such as those offered under the designations Luviquat® FC 370, FC 550, and the INCI name Polyquaternium-16, as well as FC 905 and HM 552,
quaternized vinylpyrrolidone/dimethylamino ethyl methacrylate, for example vinylpyrrolidone/dimethylaminoethyl methacrylate methosulfate copolymer that is marketed under the commercial names Gafquat® 755 N and Gafquat® 734 by the GAF company, USA, and the INCI name Polyquaternium-11,
quaternized poly(vinylalcohol),
and the polymers known by the names Polyquaternium-2, Polyquaternium-17, Polyquaternium-18, and Polyquaternium-27, having quaternary nitrogen atoms in the main polymer chain,
vinylpyrrolidone/vinylcaprolactam/acrylate terpolymers such as those having acrylic acid esters and acrylic acid amides as a third monomer module, and offered commercially e.g. under the designation Aquaflex® SF 40.

Amphoteric polymers according to the present invention are those polymerizates in which a cationic group derives from at least one of the following monomers:

(i) monomers having quaternary ammonium groups of the general formula (Mono1)

R¹—CH═CR²—CO—Z—(CₙH₂ₙ)—N⁽⁺⁾R²R³R⁴ (Mono1),
in which R¹ and R² mutually independently denote hydrogen or a methyl group and R³, R⁴, and R⁵ mutually independently denote alkyl groups having 1 to 4 carbon atoms, Z denotes an NH group or an oxygen atom, n is an integer from 2 to 5, and A⁽⁻⁾ is the anion of an organic or inorganic acid,
   (ii) monomers having quaternary ammonium groups of the general formula (Mono2)

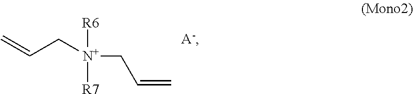

in which R⁶ and R⁷ mutually independently denote a (C₁ to C₄) alkyl group, in particular a methyl group, and
A⁻ is the anion of an organic or inorganic acid,
   (iii) monomeric carboxylic acids of the general formula (Mono3)

R⁸—CH═CR⁹—COOH (Mono3),
in which R⁸ and R⁹ mutually independently are hydrogen or methyl groups.

Those polymerizates in which the monomers used are of type (i) in which R³, R⁴, and R⁵ are methyl groups, Z is an NH group, and A⁽⁻⁾ is a halide, methoxysulfate, or ethoxysulfate ion, are particularly preferred; acrylamidopropyltrimethylammonium chloride is a particularly preferred monomer (i). Acrylic acid is preferably utilized as monomer (ii) for the aforesaid polymerizates.

Particularly preferred amphoteric polymers are copolymers of at least one monomer (Mono1) or (Mono2) with the monomer (Mono3), in particular copolymers of monomers (Mono2) and (Mono3). Amphoteric polymers used very particularly preferably according to the present invention are copolymerizates of diallyldimethylammonium chloride and acrylic acid. These copolymerizates are marketed under the INCI name Polyquaternium-22, inter alia with the commercial name Merquat® 280 (Nalco).

Furthermore, the amphoteric polymers according to the present invention can additionally include, besides a monomer (Mono1) or (Mono2) and a monomer (Mono3), a monomer (Mono4)

(iv) monomeric carboxylic acid amides of the general formula (Mono4),

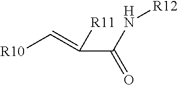

in which R¹⁰ and R¹¹ mutually independently are hydrogen or methyl groups, and R¹² denotes a hydrogen atom or a (C₁ to C₈) alkyl group.

Amphoteric polymers based on a comonomer (Mono4) that are used very particularly preferably according to the present invention are terpolymers of diallyldimethylammonium chloride, acrylamide, and acrylic acid. These copolymerizates are marketed under the INCI name Polyquaternium-39, inter alia with the commercial name Merquat® Plus 3330 (Nalco).

Amphoteric polymers can in general be used according to the present invention both directly and in a salt form that is obtained by neutralizing the polymerizates, for example using an alkali hydroxide.

The cationic polymers recited above can be used individually or in any combinations with one another, quantities between 0.01 and 10 wt %, preferably quantities from 0.01 to 7.5 wt %, and very particularly quantities from 0.1 to 5.0 wt % being included. The best results of all are obtained with quantities from 0.1 to 3.0 wt %, based in each case on the total composition of the respective agent.

For aesthetic reasons, “clear” products are often preferred by consumers Hair treatment agents preferred according to the present invention are therefore characterized in that they are transparent or translucent.

“Transparent or translucent” is understood, in the context of the present invention, as a composition that has an NTU value below 100. The NTU value (nephelometric turbidity unit) is a unit used in water treatment for turbidity measurements in liquids. It is the unit of the turbidity of a liquid measured with a calibrated nephelometer.

The agents according to the present invention can furthermore include a 2-pyrrolidinone-5-carboxylic acid and derivatives thereof (J). The sodium, potassium, calcium, magnesium, or ammonium salts, in which the ammonium ion carries, beside hydrogen, one to three C₁ to C₄ alkyl groups, are preferred. The sodium salt is very particularly preferred. The quantities used in the agents according to the present invention are preferably 0.05 to 10 wt % based on the total agent, particularly preferably 0.1 to 5, and in particular 0.1 to 3 wt %.

It can additionally prove to be advantageous if penetration adjuvants and/or swelling agents (M) are included in the compositions according to the present invention. To be included thereamong are, for example, urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as, for example, 1,2-propanediol, 1,2-pentanediol, 1,2-hexanediol, 1,2-dodecanediol, 1,3-propanediol, 1,6-hexanediol, 1,5-pentanediol, 1,4-butanediol.

In addition, short-chain carboxylic acids (N) can advantageously assist the active agent complex (A). “Short-chain carboxylic acids” and derivatives thereof are understood for purposes of the invention as carboxylic acids that can be saturated or unsaturated and/or straight-chain or branched or cyclic and/or aromatic and/or heterocylic, and have a molecular weight of less than 750. Saturated or unsaturated straight-chain or branched carboxylic acids having a chain length from 1 to 16 carbon atoms in the chain can be preferred for purposes of the invention; those having a chain length from 1 to 12 carbon atoms in the chain are very particularly preferred.

The short-chain carboxylic acids for purposes of the invention can comprise one, two, three, or more carboxy groups. Carboxylic acids having multiple carboxy groups, in particular di- and tricarboxylic acids, are preferred for purposes of the invention. The carboxy groups can be present entirely or partly as an ester, acid anhydride, lactone, amide, imidic acid, lactam, lactim, dicarboximide, carbohydrazide, hydrazone, hydroxam, hydroxime, amidine, amidoxime, nitrile, phosphonic ester, or phosphate ester. The carboxylic acids used according to the present invention can of course be substituted along the carbon chain or ring structure. To be included among the substituents of the carboxylic acids used according to the present invention are, for example, C1 to C8 alkyl, C2 to C8 alkenyl, aryl, aralkyl, and aralkenyl, hydroxymethyl, C2 to C8 hydroxyalkyl, C2 to C8 hydroxyalkenyl, aminomethyl, C2 to C8 aminoalkyl, cyano, formyl, oxo, thioxo, hydroxy, mercapto, amino, carboxy, or imino groups. Preferred substituents are C1 to C8 alkyl, hydroxymethyl, hydroxy, amino, and carboxy groups. Substituents in the α-position are particularly preferred. Very particularly preferred substituents are hydroxy, alkoxy, and amino groups, wherein the amino function can optionally be further substituted with alkyl, aryl, aralkyl, and/or alkenyl residues. In addition, the phosphonic esters and phosphate esters are likewise preferred carboxylic acid derivatives.

A further subject of the present invention is a method for treating keratinic fibers, in particular human hair, in which method a hair treatment agent according to the present invention is applied onto keratinic fibers and either is left there until the hair is next washed (so-called “leave-on” product) or is rinsed off after a contact time from 30 to 900 seconds (so-called “rinse-off” product).

A further subject of the present invention is therefore a method for hair treatment in which a hair treatment agent according to the present invention is applied onto the hair and is rinsed out again after a contact time from 5 seconds to 15 minutes.

A further subject of the present invention is therefore a method for hair treatment in which a hair treatment agent according to the present invention is applied onto the hair and is left there until the hair is next washed.

The statements made regarding the agents according to the present invention apply mutatis mutandis with regard to preferred embodiments of the methods according to the present invention.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair-conditioning agent that includes, based in each case on its total weight, in a cosmetic carrier,
a) at least one esterquat according to formula (Tkat1-2) 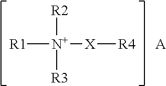
in a total quantity from 0.01 to 20.0 wt %,
wherein the residues R1, R2, and R3 are each mutually independent and can be identical or different, and the residues R1, R2, and R3 signify:
a branched or unbranched alkyl residue having 1 to 4 carbon atoms, in particular methyl, ethyl, propyl, isopropyl, butyl, tert-butyl, or isobutyl, particularly preferably methyl,
the residue (—X—R4), provided that at most two of the residues R1, R2, or R3 can denote this residue;
the residue (X—R4) is included at least once and at most twice, particularly preferably twice, and X denotes:
—(CH2)n-, where n=1 to 20, preferably n=1 to 10, and particularly preferably n=1 to 5 and most preferably n=2, and R4 denotes
R6-O—CO—, in which R6 is a saturated or unsaturated, branched or unbranched, or a cyclic saturated or unsaturated alkyl residue having 6 to 30 carbon atoms, which can include at least one hydroxy group, preferably a saturated, branched or unbranched alkyl residue having 12 to 30 carbon atoms, particularly preferably a saturated unbranched alkyl residue having 12 to 30, preferably 12 to 24 carbon atoms,
and A denotes a physiologically acceptable organic or inorganic anion selected from the halide ions fluoride, chloride, bromide, iodide, sulfates of the general formula RSO₃⁻, in which R has the meaning of a saturated or unsaturated alkyl residue having 1 to 4 carbon atoms, or anionic residues of organic acids such as maleate, fumarate, oxalate, tartrate, citrate, lactate, or acetate,
and
b) at least one care-providing substance(s) in a total quantity of said care-providing substances from 0.001 to 10 wt %, preferably 0.005 to 7.5 wt %, particularly preferably 0.01 to 5 wt %, and in particular 0.05 to 2.5 wt %, wherein the care-providing substance(s) are selected from the group
i. L-carnitine and/or salts thereof, and/or
ii. taurine and/or salts thereof, and/or
iii. vitamins and vitamin precursors, and/or
iv. niacinamide, and/or
v. ubiquinone, and/or
vi. ectoin.

2. The agent according to claim 1, wherein the esterquat is selected from Dilauroylethyl Dimonium Chloride, Dimyristoylethyl Dimonium Chloride, Dipalmitoylethyl Dimonium Chloride, Distearoylethyl Dimonium Chloride, Diarachidoylethyl Dimonium Chloride, and Dibehenoylethyl Dimonium Chloride, or the respective methosulfate compound.

3. The agent according to claim 1, wherein the agent includes at least one silicone selected from the group consisting of dimethicones, cyclomethicones, amodimethicones, and dimethiconols.

4. The agent according to claim 1, wherein the agent includes at least one silicone selected from the group consisting of dimethicones and cyclomethicones.

5. The agent according to claim 1, wherein the agent furthermore includes at least one further quaternary compound.

6. The agent according to claim 5, characterized in that the further quaternary compound is at least one quaternary ammonium compound in a total quantity from 0.1 to 10.0 wt % based on the weight of the total composition, selected from the group consisting of:
i. quaternary imidazolines of formula (Tkat2), 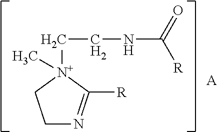
in which the residues R mutually independently each denote a saturated or unsaturated, linear or branched hydrocarbon residue having a chain length from 8 to 30 carbon atoms, and A denotes a physiologically acceptable anion,
ii. amines and/or cationized amines,
iii. poly(methacryloyloxyethyltrimethylammonium) compounds,
iv. quaternized cellulose derivatives,
v. cationic alkylpolyglycosides,
vi. cationized honey,
vii. cationic guar derivatives,
viii. chitosan,
ix. polymeric dimethyldiallylammonium salts and copolymers thereof with esters and amides of acrylic acid and methacrylic acid,
x. copolymers of vinylpyrrolidone with quaternized derivatives of dialkylaminoalkyl acrylate and methacrylate,
xi. vinylpyrrolidone-vinylimidazolium methoehloride copolymers,
xii. quaternized poly(vinyl alcohol),
xiii. Polyquaternium-74,
and mixtures thereof.

7. The agent according to claim 5, wherein the further quaternary compound is at least one member selected from the group consisting of:
i. amines and/or cationized amines,
ii. poly(methacryloyloxyethyltrimethylammonium) compounds,
iii. quaternized cellulose derivatives,
iv. Polyquaternium-74,
and mixtures thereof.

8. A method for treating hair, comprising:
applying the agent according to claim 1 onto the hair, and
rinsing the agent out again after a contact time from 0 to 45 minutes.

9. A method for treating hair, comprising:
applying the agent according to claim 1 onto the hair and is leaving the agent there until it is next washed.
